(19) United States
(12) Patent Application Publication
(10) Pub. No.: US 2012/0253261 A1
(43) Pub. Date: Oct. 4, 2012

(54) SYSTEMS AND METHODS FOR OPTOGENETIC MODULATION OF CELLS WITHIN A PATIENT

(75) Inventors: CHRISTOPHER POLETTO, NORTH OAKS, MN (US); JONATHON E. GIFTAKIS, MAPLE GROVE, MN (US); WILLIAM F. KAEMMERER, EDINA, MN (US)

(73) Assignee: MEDTRONIC, INC., MINNEAPOLIS, MN (US)

(21) Appl. No.: 13/074,808

(22) Filed: Mar. 29, 2011

(51) Int. Cl. A61M 37/00

(52) U.S. Cl. 604/20

(57) ABSTRACT

Cells within a patient are optogenetically modulated to treat various neurological disorders. In one example, a method includes delivering a viral vector including a genetic agent encoding for one or more light-sensitive proteins to a delivery site within a patient. The viral vector includes retrograde and/or anterograde transport properties such that the viral vector is configured to transduce the genetic agent into cells at the delivery site and into cells in a plurality of sites proximal and remote to the delivery site. A bioelectrical signal(s) related to a neurological condition of the patient is sensed, e.g. using an implanted electrode. Optical stimulation is delivered to cells transduced with the genetic agent by the viral vector to treat the neurological condition of the patient.

### STATEMENT REGARDING SEQUENCE LISTING

The Sequence Listing in accordance with 37 C.F.R. §§1.821-1.824 associated with this application is provided in text format in lieu of a paper copy, and is hereby incorporated by reference into the specification in its entirety. The name of the text file containing the Sequence Listing is “SEQUENCELISTING.txt.” The text file is 4 KB, was created on Mar. 29, 2011, and is being submitted electronically via EFS-Web.

### BACKGROUND

Electrical stimulation of neural tissue serves as the core of many neurological therapies, and can provide relief for a variety of disorders, improving the quality of life for many patients. In some cases, electrical stimulation may be characterized by a lack of specificity in the excitation of neural tissue. In particular, it can be difficult to stimulate a specific, localized neural population due to constraints on electrode geometry and placement. For example, the area of stimulation may be dictated by electrode size, which can be generally orders of magnitude greater than the cellular targets of interest. In some cases, this may lead to overexciting cellular networks and or inefficient stimulation, and may result in stimulation of non-target cells.

In addition, inhibitory stimuli through the use of electrical coupling can be challenging and may involve stimulation waveforms with much lower efficiency than those for activation. The presence of electrodes in tissue may also place limitations on electromagnetic field exposure from electromagnetic sources such as magnetic resonance imaging (MRI) and electrosurgery devices. In addition, electrical stimulation can undermine the ability to sense underlying electrical neural activity simultaneously with delivery of electrical stimulation. For example, electrical stimulation currents flowing through the tissue that are necessary to achieve a localized current density high enough to depolarize a cell or axon can mask the bioelectrical activity to be sensed.

### SUMMARY

Examples according to this disclosure are directed to optogenetic modulation of target cells within a patient to provide therapy for one or more of a variety neurological disorders. In one example, a method includes delivering a viral vector including a genetic agent encoding for one or more light-sensitive proteins to a delivery site within a patient, sensing a bioelectrical signal related to a neurological condition of the patient, and delivering optical stimulation to one or more cells transduced with the genetic agent by the viral vector based on the sensed bioelectrical signal. The viral vector includes at least one of retrograde or anterograde transport properties such that the viral vector is configured to transduce the genetic agent into cells at the delivery site and into cells in a plurality of sites proximal and remote to the delivery site

In another example, a medical system includes a biological vector delivery device, a sensor, and an optical stimulator. The biological vector delivery device is configured to deliver a viral vector including a genetic agent encoding for one or more light-sensitive proteins to a delivery site within a patient. The viral vector includes at least one of retrograde or anterograde transport properties such that the viral vector is configured to transduce the genetic agent into cells at the delivery site and into cells in a plurality of sites proximal and remote to the delivery site. The sensor is configured to sense a bioelectrical signal related to a neurological condition of the patient. The optical stimulator is configured to deliver light to one or more of the cells transduced with the genetic agent by the viral vector based on the bioelectrical signal sensed by the sensor.

In another example, a system includes means for delivering a viral vector including a genetic agent encoding for one or more light-sensitive proteins to a delivery site within a patient, means for sensing a bioelectrical signal related to a neurological condition of the patient, and means for delivering optical stimulation to one or more cells transduced with the genetic agent by the viral vector based on the bioelectrical signal. The viral vector includes at least one of retrograde or anterograde transport properties such that the viral vector is configured to transduce the genetic agent into cells at the delivery site and into cells in a plurality of sites proximal and remote to the delivery site.

In another example, a method includes delivering a viral vector including a genetic agent encoding for one or more light-sensitive proteins to a delivery site in the hippocampus within a first hemisphere of the brain of a patient. The viral vector includes at least one of retrograde or anterograde transport properties such that the viral vector is configured to transduce the genetic agent into cells at the delivery site and into cells in a plurality of sites proximal and remote to the delivery site. The method also includes sensing a bioelectrical signal related to epilepsy and delivering optical stimulation to one or more cells transduced with the genetic agent in the cerebral cortex of the brain of the patient based on the sensed bioelectrical signal.

In another example, a system includes a biological vector delivery device, a sensor, and an optical stimulator. The biological vector delivery device is configured to deliver a viral vector including a genetic agent encoding for one or more light-sensitive proteins to a delivery site in the hippocampus within a first hemisphere of the brain of a patient. The viral vector includes at least one of retrograde or anterograde transport properties such that the viral vector is configured to transduce the genetic agent into cells at the delivery site and into cells in a plurality of sites proximal and remote to the delivery site. The sensor is configured to sense a bioelectrical signal related to epilepsy. The optical stimulator is configured to deliver light to one or more cells transduced with the genetic agent in the cerebral cortex of the brain of the patient based on the bioelectrical signal sensed by the sensor.

The details of one or more examples are set forth in the accompanying drawings and the description below. Other features, objects, and advantages of examples according to this disclosure will be apparent from the description and drawings, and from the claims.

### BRIEF DESCRIPTION OF DRAWINGS

FIG. 1 is a conceptual diagram illustrating an example therapy system that includes an implantable stimulator coupled to optical fibers for optical stimulation.

FIGS. 2A and 2B are coronal sections of a human brain illustrating transduction of a genetic agent by a viral vector with retrograde transport properties.

FIG. 3 is a block diagram illustrating various components of an example configuration of the implantable stimulator of FIG. 1.

FIG. 4 is a conceptual diagram illustrating another example therapy system that includes an implantable stimulator coupled to optical electrodes on an electrode array.

FIG. 5 is a conceptual diagram illustrating another example therapy system that includes an implantable stimulator coupled to optical fibers for optical stimulation and catheters for delivery of a therapeutic agent.

FIG. 6 is a conceptual diagram illustrating another example therapy system that includes an implantable stimulator coupled to optical fibers.

FIG. 7 is a flow chart illustrating a method of optogenetically modulating a target population of cells within a patient.

FIGS. 8-26 show results from several animal studies conducted to test widespread transduction of DNA by adeno-associated virus (AAV) vectors in cells of subject animals' brains, including brain sections from mice and sheep injected with AAV vectors, immunostained to show cell transduction and quantitative data produced from the studies.

### DETAILED DESCRIPTION

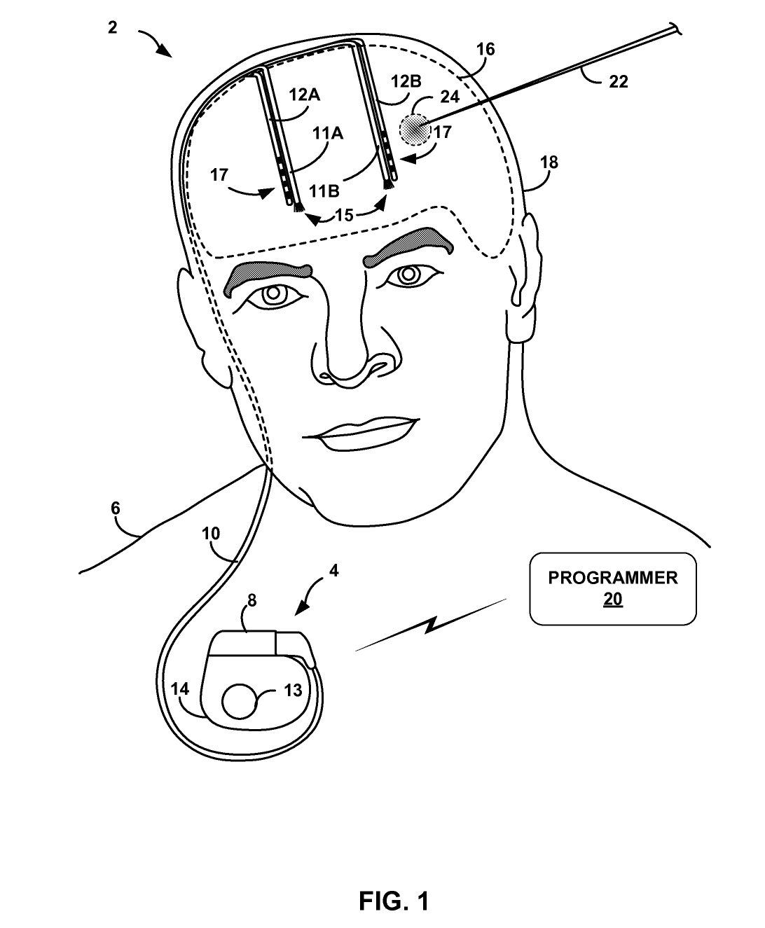

FIG. 1 is a conceptual diagram illustrating an example system 2 that may be used to deliver stimulation therapy to patient 6. Patient 6 ordinarily, but not necessarily, will be a human. Therapy system 2 includes implantable stimulator 4, lead body 10, and programmer 20. Implantable stimulator 4 includes header 8, housing 14, and housing electrode 13. In some examples, lead body 10 may include a bundle of a number of different components connected to stimulator 4 and extending to a target tissue site within patient 6. In the example of FIG. 1, lead body 10 bundles first optical fiber 11A and second optical fiber 11B (collectively referred to as “optical fibers 11”), as well as first electrical lead 12A and second electrical lead 12B (collectively referred to as “electrical leads 12”). Lead body 10 is connected to header 8 of stimulator 4, thereby connecting optical fibers 11 and electrical leads 12 to the stimulator. Housing electrode is connected to housing 14. Programmer 20 may be employed or implemented as either a clinician or patient programmer and may be a handheld computing device that permits users, e.g. a clinician or patient to communicate wirelessly with stimulator 4 implanted within patient 6.

System 2 also includes a vector delivery device, which, in the example of FIG. 1, is micropipette 22 configured to deliver a biological vector to target site 24 intracranially. As discussed below, the vector delivered to target site 24 by micropipette 22 is configured to transduce genetic agents encoding for light-sensitive proteins into cells in a plurality of regions of brain 16 of patient 6 proximal and remote to target site 24. The genetic agents thus transduced to cells in a plurality of regions express the light sensitive proteins and may be optically stimulated by stimulator 4 to modulate the behavior of the cells via control of the light-sensitive proteins, e.g. by opening a channel or driving a pump to raise or lower the membrane potential of a nerve cell.

Implantable stimulator 4 may be configured to deliver optical stimulation, such as light 15, to patient 6 via implantable optical fibers 11. The terms “light” or “optical light” as used herein may refer to electromagnetic radiation having a wavelength and intensity that has a physiologically measurable effect and may include visible light, infrared light, and ultraviolet light. In some examples, light that may be used to provide the optical stimulation of system 2 may include visible light having a wavelength of between about 380 nm and about 750 nm, infrared light having a wavelength of between about 700 nm and about 300 μm, and ultraviolet light having a wavelength between about 10 nm and about 400 nm. For example, first optical fiber 11A may deliver visible light having a certain wavelength and intensity, and second optical fiber 11B may deliver visible light having the same wavelength and intensity, or a different wavelength at the same intensity, or the same wavelength and a different intensity, or the second optical fiber 11B may deliver non-visible light, such as infrared or ultraviolet light. The fibers 11A and 11B may be coupled to the same light source or different light sources. In some cases, a single light source may be optically multiplexed across the fibers 11A, 11B to deliver light via the different fibers at different times. In some examples, the light source may deliver light via both fibers 11A, 11B simultaneously. The light delivered via one optical fiber 11A may be the same as the light delivered via another optical fiber 11B, e.g., in terms of characteristics or parameters such as wavelength, amplitude, pulse width or pulse rate. Alternatively, the light delivered via the optical fibers 11A, 11B may have different characteristics or parameters.

The implantable optical fibers 11A, 11B may be deployed to a target site as part of one or more bundles of optical fibers, such as implantable optical fibers 11 bundled in lead body 10, or separately. In some cases, stereotactic or other positioning techniques may be used to precisely position the optical fibers with respect to target tissue sites. The optical stimulation may be in the form of optical light of a particular wavelength and may be delivered as pulses, e.g., with a defined pulse width and pulse rate. Various parameters of the pulses may be defined by a stimulation program. The pulses may be delivered substantially continuously for a relatively long period of time, such as several seconds or more, or in pulse bursts, segments, or patterns, and may be delivered alone or in combination with pulses defined by one or more other stimulation programs. Although FIG. 1 shows a fully implantable stimulator 4, techniques described in this disclosure may be applied to external stimulators having optical fibers deployed via percutaneously implantable leads.

As illustrated in FIG. 1, system 2 may include sense electrodes deployed within patient 6, such as implantable sense electrodes 17 implanted on leads 12A and 12B alongside optical fibers 11 and/or housing electrode 13 located on housing 14, i.e., “can” or “case,” of implantable stimulator 4. Leads 12A, 12B may be implanted side-by-side with optical fibers 11A, 11B, respectively, and fastened or formed together, such as bundled via lead body 10. In other examples, leads and associated sense electrodes may be formed in a common lead body with one or more optical fibers, such as a conductor and one or more electrodes placed on a lead sheath that covers an optical fiber. In some examples, an electrical conductor and optical fiber can run axially along the lead, while in another example an electrical conductor may be wound in a coil that runs along the lead while one or more optical fibers extend through the middle of the coil. In other examples, implantable stimulator 4 may be coupled to one or more leads which may or may not be bifurcated. In such examples, the leads may be coupled to implantable stimulator 4 via a common lead extension or via separate lead extensions.

The sense electrodes associated with electrical leads 12 connected to stimulator 4 may detect various types of bioelectric signals, including local field potentials (LFP) of brain tissue, energy spectra in different bands, such as alpha, beta, or gamma bands of brain activity, and electrical signals associated with electrocorticography (ECoG) or electroencephalography (EEG). In one example, sense electrodes on leads 12 may be employed by stimulator 4 to predict the onset or detect the occurrence of a seizure related to or caused by a neurological condition of patient 6. For example, stimulator 4 may be configured to employ leads 12 to sense one or more bioelectrical signals, e.g. LFP, ECoG, and/or EEG in order to predict the onset or detect the occurrence of an epileptic seizure. Upon predicting or detecting the seizure, stimulator 4 may deliver therapy to brain 16 of patient 6 to help mitigate the effects of the seizure or, in some cases, prevent the onset of the seizure or manifestations of the seizure that are perceived by the patient.

Other sensors may also be included within or on housing 14 or external to housing 14 within patient, including an accelerometer or other posture sensor and a pressure sensor. In some examples, in addition to sensing bioelectric signals or as an alternative, the sense electrodes could be selectively used to deliver electrical stimulation, such that the implantable stimulator may deliver optical stimulation and electrical stimulation on, e.g., a selective basis. For example, optical or electrical stimulation could be delivered at different times or at the same time independently of one another or on a coordinated basis. In such an example, the stimulator may include a stimulation engine configured to deliver the electrical stimulation in the form of pulses or one or more different types of continuous waves.

Optical stimulation of target tissue within patient 6 by stimulator 4 via fibers 11 may be employed for optogenetic modulation of a target population of cells, such as, for example, a particular area of neurons within the brain or spinal cord. Optogenetic modulation or stimulation may refer to the combination of transduction of a genetic agent encoding for light-sensitive protein(s) into target cells within the body of a patient and optical stimulation of the transduced cells to modulate the behavior of the cells via control of the light-sensitive protein(s). For example, a biological vector may be employed to transduce neurons of patient 6 with a nucleic acid encoding for light-sensitive protein(s), channels or pumps, referred to herein as “opsins.” The transduced neurons may express the protein(s), which may refer to the neurons using the new nucleic acid as instructions to produce the protein. The transduced neurons may then be optically stimulated by stimulator 4 via fibers 11 to control the light-sensitive proteins in the walls of the neurons and thereby modulate the membrane potential, e.g. raise or lower the membrane potential of the neurons. Modulating the membrane potential of target neurons within patient 6 may be employed to selectively silence or activate the cells to treat a number of neurological conditions, including, e.g., depression, dementia, obsessive-compulsive disorder and movement disorders, such as Parkinson's disease, spasticity, epilepsy, and dystonia.

It has been discovered that small, double-stranded ribonucleic acid (RNA) or relatively small pieces of deoxyribonucleic acid (DNA) can be used to selectively silence the expression of individual genes in cells, including brain cells. This discovery has opened up new possibilities for treating brain disorders, including invasive brain cancer, Alzheimer's disease, and symptoms of Parkinson's disease. Realizing the clinical potential of these possibilities requires the ability to deliver DNA or RNA to wide regions of the human brain. However, reaching more than one region of the brain without highly invasive procedures can be challenging. Therefore, examples according to this disclosure include techniques for efficiently delivering genetic agents to a plurality of regions of a patient's brain through minimally invasive surgical techniques. In particular, the disclosed examples are directed to delivering genetic agents encoding for light-sensitive proteins to a single region of a patient's brain, in which the vector delivering the genetic agents is configured to transduce the agents into cells in a plurality of regions of the brain proximal and remote to the delivery region. The disclosed examples also include optically stimulating one or more regions of the brain to treat a neurological condition of the patient by modulating the behavior of the transduced cells via control of the light-sensitive proteins in the cells.

As noted above, one facet of optogenetic stimulation includes the transduction of a genetic agent encoding for light-sensitive protein(s) to target cells within the body of a patient. One biological vector that may be employed to transduce cells of a patient with a nucleic acid encoding for light-sensitive protein(s), channels or pumps, includes a viral vector. While a number of types of viral vectors are capable of transducing cells with a nucleic acid encoding for light-sensitive proteins, examples according to this disclosure employ viral vectors with retrograde and/or anterograde transport properties. In particular, examples according to this disclosure employ viral vectors that are capable of retrograde and/or anterograde transport after initial delivery within a patient to sites other than the delivery site such that the nucleic acid encoding for light-sensitive proteins is transduced to cells not only at the delivery site, but also to cells in a plurality of regions within the patient proximal and remote to the delivery site.

A number of viral vectors with retrograde and/or anterograde transport properties may be employed in examples according to this disclosure. In one example, an adeno-associated viral (AAV) vector is capable of transduction of a genetic agent encoding for light-sensitive protein(s) to cells not only at a delivery site within a patient at which the vector is initially delivered, but also to cells in a plurality of regions within the patient proximal and remote to the delivery site. Different types of AAV vectors may be employed in examples according to this disclosure. In some examples, AAV vectors may include single-stranded DNA (rAAV) that requires host-cell synthesis of the complementary DNA strand for transduction. However, in other examples, an AAV vector may include double-stranded DNA, or dimeric inverted repeat DNA molecules. Dimeric, or self-complementary AAV (scAAV) may be capable of spontaneously self-annealing, alleviating the requirement for host-cell DNA synthesis.

In addition to single-stranded DNA rAAV and double-stranded or dimeric inverted repeat DNA scAAV, different AAV vectors may also be selected based on different serotypes. AAV serotypes are characterized by different compositions of an outer protein coat of the virus, which is referred to as the capsid. The composition of the capsid of the particular AAV will define the serotype of the vector. AAV serotypes 2 and 9 (AAV2 and AAV9, respectively) are two examples of AAV vectors that may be employed in examples according to this disclosure. As described in greater detail below, AAV9 may be useful because of its ability to cross the blood-brain barrier and therefore facilitate intravenous delivery of the vector to the patient.

In another example according to this disclosure, a herpes simplex viral (HSV) vector is employed to transduce a genetic agent encoding for light-sensitive proteins to cells not only at the delivery site, but also to cells in a plurality of regions within the patient proximal and remote to the delivery site. In other examples, one of a number of different types of lentivirus vectors may be employed to transduce a genetic agent encoding for light-sensitive protein(s) to target cells within a patient.

A variety of techniques may be employed to deliver the viral vector with retrograde and/or anterograde transport properties and including the genetic agent encoding for light-sensitive protein(s). In one example, the viral vector is metered by an external infusion pump and delivered to the patient by percutaneous injection. For example, an AAV vector may be injected intracranially using stereotactic coordinates, a micropipette and an automated pump for precise delivery of AAV to a desired region within a patient's brain with minimal damage to tissue surrounding the delivery site. In another example, a viral vector may be delivered to a target site within a patient intravenously. For example, an AAV vector may be delivered to sites within the brain of a patient via the carotid artery (CA).

In applications involving the brain, one advantage of some viral vectors over others may be their ability to cross the blood-brain barrier and thus be delivered intravenously versus via intracranial injection. An example of a viral vector that may be capable of crossing the blood-brain barrier is described in Duque S, Joussemet B, Riviere C, Marais T, Dubreil L, Douar A M, Fyfe J, Moullier P, Colle M A, Barkats M. “Intravenous administration of self-complementary AAV9 enables transgene delivery to adult motor neurons.” Mol Ther. 2009. July; 17(7):1187-96. Epub 2009 Apr. 14. PubMed PMID: 19367261; PubMed Central PMCID: PMC2835208. Duque et al. (2009) describes the ability of scAAV9 to not only deliver DNA to the central nervous system of neonatal mice (in which the blood-brain barrier has not yet fully formed), but also to deliver DNA to the spinal cord and some brain cells of adult mice and adult cats, upon intravenous delivery. As explained above, AAV9 differs from previous AAV vectors in that it has a unique outer protein coat, or capsid, which may enable the AAV9 vector, unlike others, to enter the brain from the vasculature, crossing the blood-brain barrier and delivering DNA widely throughout the brain.

Whatever viral vector is employed and whatever technique is used to deliver the vector to a target site within a patient, a variety of genetic agents encoding for light-sensitive protein(s), referred to herein as “opsins,” may be delivered by the vector. Delivering the genetic agents encoded for one of a number of appropriate opsins permits modulation of the transduced cells via optical stimulation. For example, the activation or inhibition of neurons transduced with DNA encoding for an opsin may be controlled by directing light of varying wavelengths and intensities at the cells. The devices and techniques described in this disclosure may be used in conjunction with any of a variety of opsins or other materials effective in supporting excitation, inhibition or other desired effects on the target tissue, e.g. of target neurons. In one example, a first opsin or set of opsins, also referred to as a light-activated cation channel proteins (or “LACC”), comprises the protein, or portions of the protein Channelrhodopsin-2 (ChR2). The opsins employed in examples according to this disclosure may also cause the modulation of the flow of anions such as chloride across a membrane when activated by light. In one example, a second opsin or set of opsins, also referred to as an anion pump, may comprise the protein, or portions of the protein, halorhodopsin (NpHR). A number of other example opsins may be employed including, e.g., archaerhodopsin-3 from Halorubrum sodomense (Arch), archaerhodopsin from Halorubrum strain TP009 (ArchT), and a blue-green light-drivable proton pump from the fungus Leptosphaeria maculans (Mac) (see Chow B Y, Han X, Dobry A S, Qian X, Chuong A S, Li M, Henninger M A, Belfort G M, Lin Y, Monahan P E, Boyden E S. High-performance genetically targetable optical neural silencing by light-driven proton pumps. Nature. 2010 Jan. 7; 463(7277):98-102. PubMed PMID: 20054397; PubMed Central PMCID: PMC2939492).

In some examples according to this disclosure, the viral vector may deliver a genetic agent encoding for one opsin to target cells within the patient, while, in other examples, the vector may deliver a genetic agent encoding for more than one opsin. In one example according to this disclosure, a first opsin may be employed as an activating or exciting opsin that, when exposed to a specific wavelength of light or range of wavelengths, causes the target neuron membrane to become permeable to specific anions or cations flowing into the neuron, which depolarizes the neuron, also referred to as activating the neuron, and causes a neural spike. A second opsin may be employed in addition to or in lieu of the first activating opsin as an inhibiting opsin that, when exposed to a different wavelength of light or range of wavelengths, acts to hyperpolarize the neuron, also referred to as inhibiting or deactivating the neuron, to counteract the inward cation permeability of the target neuron.

An example of a first opsin that may be employed as an activating opsin is ChR2, which is activated to provide a cation-permeable channel that activates a target neuron. In one example, the cation-permeable channel of the ChR2 opsin may activate the target neuron when exposed to light having a wavelength between about 420 nm and about 500 nm, such as between about 450 nm and about 495 nm, or in one example about 470 nm, and with an intensity of between about 0.5 mW/mm² and about 10 mW/mm², such as between about 1 mW/mm² and about 5 mW/mm², and in one example about 2.4 mW/mm². In one example, a ChR2 opsin employed in examples according to this disclosure is activated by blue light having a wavelength of between about 450 nm and about 495 nm, such as between about 450 nm and about 470 nm. In some examples, ChR2 opsins may be exposed to light with such characteristics for a pulse of between about 1 ms and about 1 second, such as between 5 ms and about 50 ms, and in one example about 10 ms. The ChR2 opsin may hold its activated state and slowly deactivates with a probability window of several seconds. In one example, a ChR2 opsin may also be deactivated or “switched off” by exposure to light with a second wavelength of light. In one example, a ChR2 opsin may be deactivated by illumination with a green light having a wavelength of between about 495 nm and about 570 nm, such as between about 510 nm and about 550 nm, and in one example about 535 nm, with an intensity of between about 0.5 mW/mm² and about 10 mW/mm², such as between about 1 mW/mm² and about 5 mW/mm², and in one example about 2.4 mW/mm². In such examples, the ChR2 opsin may be exposed to the green light for a pulse of between about 20 ms and about 75 ms, such as between about 40 ms and about 60 ms, and in one example about 50 ms.

An example of a second opsin is NpHR, which may be activated to provide an anion pump that inhibits or deactivates a target neuron transduced with the DNA encoded for the opsin by a viral vector with retrograde and/or anterograde transport properties, e.g. AAV. In one example, an NpHR opsin is activated, thus deactivating the target neuron, when exposed to yellow light having a wavelength of between about 550 nm and about 610 nm, such as between about 570 nm and about 590 nm, and in one example about 580 nm, and with an intensity of between about 0.5 mW/mm² and about 25 mW/mm², such as between about 10 and about 21 mW/mm² in one example or between about 1 mW/mm² and about 5 mW/mm² in another example. The NpHR may only need to be exposed to light with such characteristics for between about 10 ms and about 1 second, such as between 20 ms and about 100 ms, and in one example about 40 ms. Not only can NpHR be used to inhibit the firing of the target neurons, but it can also be used to deactivate neurons that were previously activated via stimulation of, e.g., a ChR2 opsin described above. For example, if a light pulse of a first wavelength, e.g. a 470 nm, is emitted for about 10 ms to control an ChR2 opsin to activate target neurons, which can remain active for several seconds, a light pulse of a second wavelength, e.g. 535 nm, may be emitted to deactivate the ChR2, and/or a 580 nm wavelength light pulse may be emitted to activate the NpHR, thereby abruptly deactivating the target neurons. In one example, both a first opsin that activates a target neuron and a second opsin that inhibits the target neuron may be activated simultaneously or substantially simultaneously in order to modulate the threshold potential of the target neuron to treat certain conditions, such as schizophrenia.

In some examples, light delivered by stimulator 4 to control light-sensitive proteins, e.g. ChR2 or NpHR, may be delivered in pulses that are characterized, in addition to wavelength, intensity, and duration, based on different inter-pulse intervals or duty cycles. In one example, stimulator 4 may deliver light to cells expressing ChR2 opsins as a pulse with a pulse width of about 10 ms. Stimulator 4 may also deliver the 10 ms width pulse multiple times to the cells expressing ChR2 such that the light delivered by the stimulator is defined by, e.g. an inter-pulse interval. In one example, the inter-pulse interval of light delivered by stimulator 4 to control light-sensitive proteins in examples according to this disclosure may be based on the manner in which the proteins are sought to be controlled. For example, stimulator 4 may be configured to deliver light cells expressing ChR2, which hold an activated state and slowly deactivate over a period of several seconds. In the case the desired effect of stimulation is to persistently activate the target cells using the optical stimulation from stimulator 4, inter-pulse interval of the light delivered by the stimulator may be set to just less than the time it takes the ChR2 to deactivate after being activated by the previous pulse. In this manner, stimulator 4 may hold the ChR2 in the target cells active continuously for some period of time. In another example, stimulator 4 may be configured to deliver light to cells expressing ChR2, which remain active only as long as the light is stimulating the cells. In such an example, the inter-pulse interval of the light delivered by stimulator 4 may be set directly based on how long it is desired to hold the ChR2 active.

Additional information regarding the manner in which opsins modulate cell behavior, and, in particular, modulate neuron behavior, as well as the composition of some of the foregoing opsins, including ChR2 and NpHR is described in U.S. application Ser. No. 12/951,766, filed Nov. 22, 2010, which claims the benefit of U.S. Provisional Application No. 61/264,550, filed Nov. 25, 2009 and of U.S. Provisional Application No. 61/301,836, filed Feb. 5, 2010, the entire contents of all of which are incorporated herein by this reference.

In one example, an opsin expressed in a transduced cell, which is, in turn optically stimulated to modulate cell behavior may include a fusion protein including a light-activated channel protein, such as a LACC protein or anion pump protein described above. Fusion proteins can be made that will create a single protein with the combined activities of several proteins. In one example, the fusion proteins can be used to target ChR2 and/or NpHR to specific cells or regions within cells.

In one example, a fusion protein comprising a LACC protein or anion pump protein is a fusion protein that targets sub-cellular regions of the cell. The fusion proteins may target, for instance, axons, dendrites, and synapses of neurons. In one example, a PDZ (PSD-95, Dlg and ZO-1) domain is fused to ChR2 which target dendrites. In another example, Axon initial segment (AIS) domain is fused to ChR2 which targets axons.

Other fusion proteins may be used, such as are proteins combining an opsin and a fluorescent protein in order to allow for monitoring of the localization of the opsin. Example fusion proteins are those with red fluorescent protein (mCherry), yellow fluorescent protein (YFP), enhanced yellow fluorescent protein (EYFP), cyan fluorescent protein (CFP), green fluorescent protein (GFP), and woodchuck hepatitis post-transcriptional regulatory element (WPRE). These fusion proteins, such as a ChR2-mCherrry fusion protein or a NpHr-EYFP or NpHR-EYFP-WPRE fusion protein, allow for the independent stimulation of ChR2 or NpHR and the simultaneous monitoring of localization. The simultaneous stimulation and monitoring of localization can be carried out in many cell types including mammalian cells.

In one example, an opsin protein is employed that is non-toxic in the cells in which it is expressed. In one example, the opsin proteins do not perturb the basal electrical properties, alter the dynamic electrical properties, or jeopardize the prospects for cellular survival. The opsin proteins may be selected so as to not alter the membrane resistance of the cells in the absence of light, lead to apoptosis in the cells, or lead to the generation of pyknotic nuclei. In one example, in the absence of light, the presence of the opsin proteins does not alter cell health or ongoing electrical activity, at the level of subthreshold changes in voltage or in spike output, either by shunting current through leaky channels or by altering the voltage dependence of existing neuronal input-output relationships. The opsin proteins used in some examples according to this disclosure may be selected so that their presence creates no significant long-term plastic or homeostatic alterations in the electrical properties of neurons expressing the proteins.

Opsin proteins employed in the disclosed examples can be encoded for by various nucleic acids. Each amino acid in the protein is represented by one or more sets of 3 nucleic acids (codons). Because many amino acids are represented by more than one codon, there is not a unique nucleic acid sequence that codes for a given protein. A nucleic acid that codes for a particular opsin protein may be fabricated by knowing the amino acid sequence of the protein. A nucleic acid sequence that codes for a polypeptide or protein is the “gene” of that polypeptide or protein. A gene can be RNA, DNA, or other nucleic acid than will code for the polypeptide or protein. An example nucleic acid sequence for coding for a LACC comprises SEQ ID NO:1. An example nucleic acid sequence for coding for an anion pump protein comprises SEQ ID NO:3.

The codon systems in different organisms can be slightly different, and therefore where the expression of a given protein from a given organism is desired, the nucleic acid sequence can be modified for expression within that organism. In one example, the nucleic acid sequence codes for an opsin protein that is optimized for expression with a mammalian cell. In one example, a nucleic acid sequence transduced by a viral vector that codes for a particular opsin is optimized for expression in a human cell. In one example, a nucleic acid sequence that codes for a light-activated cation protein that is optimized for expression with a human cell comprises SEQ ID NO:2. In one example, a nucleic acid sequence that codes for an anion pump protein that is optimized for expression with a mammalian cell comprises SEQ ID NO:3.

The foregoing concepts related to optogenetic modulation of a target population of cells, such as, for example, a particular area of neurons within the brain or spinal cord will now be described in the context of example system 2 of FIG. 1.

In the example illustrated in FIG. 1, system 2 includes micropipette 22 configured to deliver a biological vector to target site 24 intracranially. In one example, a viral vector, such as AAV is injected intracranially using stereotactic coordinates, micropipette 22 and an automated external infusion pump (not shown in FIG. 1) connected to the micropipette for precise delivery of AAV to target site 24 within brain 16 of patient 6 with minimal damage to tissue surrounding the delivery site. In another example, a viral vector may be delivered to a target site within patient 6 intravenously. For example, an AAV vector may be delivered to sites within brain 16 of patient 6 via injection into the carotid artery (CA). In the case of intravenous delivery of the vector, a particular variety of virus may be selected, e.g. AAV9, which differs from previous AAV vectors in that it has a unique outer protein coat, or capsid, which may enable the AAV9 vector, unlike others, to enter the brain from the vasculature, crossing the blood-brain barrier and delivering DNA widely throughout the brain. Whatever the particular viral vector delivered to target site 24 by micropipette 22, or another means, the selected vector includes retrograde and/or anterograde transport properties such that it is configured to transduce genetic agents encoding for light-sensitive proteins to cells in a plurality of regions of brain 16 of patient 6 proximal and remote to target site 24 at which the vector is initially delivered. For example, in system 2 of FIG. 1, a viral vector delivered to target site 24 by micropipette 22 may be configured to transduce a genetic agent into cells proximate to the distal ends of optical fibers 11A and 11B. The genetic agents thus transduced to cells in a plurality of regions of brain 16 of patient 6, e.g. proximate to the distal ends of optical fibers 11A and 11B may be optically stimulated by stimulator 4 via fibers 11 to modulate the behavior of the cells via control of the light-sensitive proteins expressed in the transduced cells.

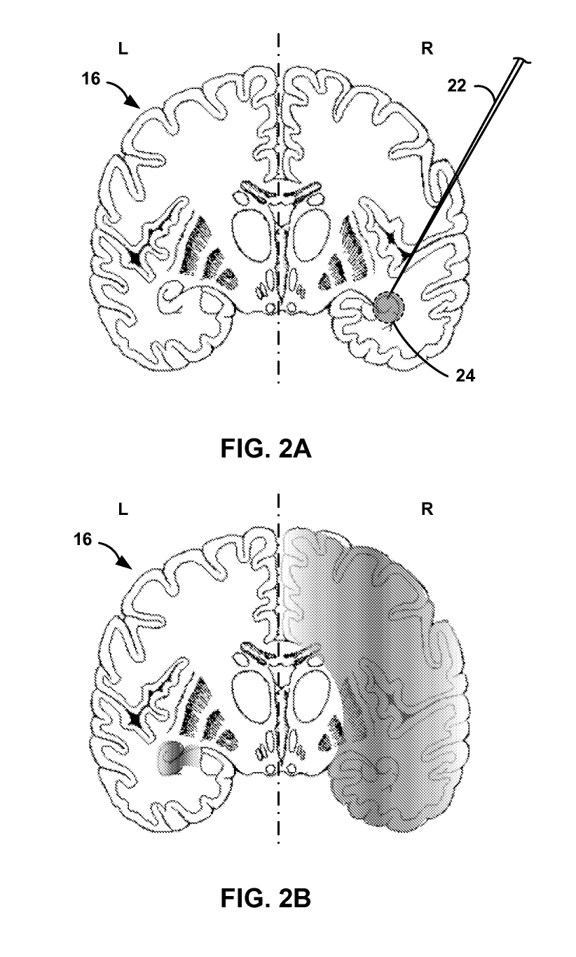

FIGS. 2A and 2B are coronal sections of a human brain illustrating transduction of a genetic agent encoded for light-sensitive proteins by a viral vector with retrograde and/or anterograde transport properties, e.g. AAV. In the example of FIG. 2A, micropipette 22 delivers a viral vector including a genetic agent encoding for light-sensitive proteins, e.g. AAV9-ArchT to target site 24 in the hippocampus of the right hemisphere of brain 16. The viral vector delivered to brain 16 includes retrograde and/or anterograde transport properties such that it is configured to transduce the genetic agent to cells in a number of regions of the brain proximal and remote to target site 24 in the hippocampus of the right hemisphere.

FIG. 2B illustrates brain 16 after some period of time after the viral vector was delivered to target site 24 in the hippocampus of the right hemisphere by micropipette 22. In FIG. 2B, the viral vector has transduced the genetic agent encoding for light-sensitive proteins into cells in a number of ipsilateral and contralateral regions in brain 16. In the example of FIG. 2B, the viral vector has transduced the genetic agent into cells in a large portion of the right hemisphere of brain 16, including the cerebral cortex. Additionally, the viral vector has transduced the agent contralaterally into cells in the hippocampus of the left hemisphere of brain 16.

Referring again to FIG. 1, implantable stimulator 4 is implanted within a subcutaneous pocket in a clavicle region of patient 6. Optical fibers 11 may be implanted using a stylet for insertion stiffness while the optical fiber is being implanted in the target tissue. For example, the stylet may allow a surgeon to easily manipulate optical fibers 11 as it is guided from the clavical region, though the neck, into cranium 18, and into brain 16 of patient. A stylet may also be used to guide optical fibers 11 to other target tissues and other treatments, such as peripheral nerve stimulation (PNS), peripheral nerve field stimulation (PNFS), deep brain stimulation (DBS), cortical stimulation (CS), pelvic floor stimulation, gastric stimulation, and the like. The stylet may be removable after insertion of optical fibers 11 so that the optical fibers are flexible after insertion such that the stylet does not interfere with chronic treatment. In one example, optical fibers 11 or lead body 10 carrying the optical fibers may include a stylet lumen for receiving the stylet and for allowing the removal of the stylet. In other examples, stimulator 4 may be external to patient 6 with a percutaneous lead body bundling optical fibers 11 and/or electrical leads 12 connected between the stimulator and the target delivery site within the patient.

Optical fibers 11, as well as leads 12, if used, may also be implanted within a desired location of brain 16 through one or more holes in cranium 18. Optical fibers 11 may be placed at any location within brain 16 such that the emitted light 15 is capable of providing optical stimulation to targeted tissue during treatment. Example locations for optical fibers 11 within brain 16 may include the pedunculopontine nucleus (PPN), thalamus, basal ganglia structures (e.g., globus pallidus, substantia nigra, subthalmic nucleus), zona inserta, fiber tracts, lenticular fasciculus (and branches thereof), ansa lenticularis, and/or the Field of Forel (thalamic fasciculus). In the case of migraines, optical fibers 11 may be implanted to provide stimulation to the visual cortex of brain 16 in order to reduce or eliminate migraine headaches afflicting patient 6. Additionally, optical fibers 11 may be implanted to provide stimulation to the cerebral cortex of brain 16 for the treatment of epilepsy. However, the target therapy delivery site may depend upon the patient condition or disorder being treated.

Stimulator 4 generates programmable optical stimulation, e.g., optical pulses with selected wavelengths and intensities, and delivers the stimulation via one or more implantable optical fibers 11. In some cases, the wavelengths and intensities of the optical pulses may be fixed, or limited to a narrow range. In other examples, the wavelengths and intensities of the optical pulses may be variable, i.e., tunable to produce a wider range of desired wavelengths and intensities. In some cases, multiple sets of one or more implantable optical fibers 11 may be provided. In the example of FIG. 1, two optical fibers 11A and 11B are each carried as part of an optical fiber bundle in lead body 10 until a distal end of the bundle is bifurcated into separate optical fiber segments 11A and 11B. Each optical fiber 11A, 11B may be a single optical fiber. Alternatively, in some examples, each optical fiber may include multiple fibers that together deliver optical stimulation. Optical fibers 11A, 11B may provide optical transmission between stimulator 4, which provides a light source for the optical stimulation, and the area of treatment, shown as the brain 16 of patient 6 in FIG. 1. Stimulator 4 provides optical stimulation by generating optical light 15 with a desired wavelength and intensity, as described in more detail below, and directing the optical light 15 into optical fiber 11 at the proximal end of the optical fiber. The optical light 15 is transmitted along optical fiber 11 until it is emitted from a distal end of optical fiber 12, as shown in FIG. 1.

In one example, stimulator 4 is configured to optically stimulate cells in brain 16 of patient 6 transduced with a genetic agent encoding for light-sensitive proteins. The optical stimulation of the transduced cells by stimulator 4 may act to modulate the behavior of the transduced cells by controlling the light-sensitive proteins expressed in the cells. For example, stimulator 4 is configured to optically stimulate cells proximate to the distal ends of fibers 11A and 11B with light 15 directed through the fibers from a light source included in the stimulator. In one example, a viral vector has been delivered to target site 24 via micropipette 22 and transduced the genetic agent into axons of cells near target site 24 and thence by retrograde and/or anterograde transport into cells that are near the distal ends of fibers 11A and 11B, which are distal to the original delivery site 24. Stimulator 4 is configured to generate optical light 15 with a desired wavelength and intensity such that the light causes the light-sensitive proteins encoded by the genetic agent in the cells within brain 16 of patient 6 to respond to the light by allowing or pumping ions into the cells thereby modulating the behavior of the cells. For example, stimulator 4 may optically stimulate neurons within brain 16 using light 15 directed to the cells through fibers 11 to activate the light-sensitive anion pump in the cells, which, in turn, lowers the resting potential of the neurons and thereby prevents the neurons from firing action potentials. Such optical stimulation of neurons within brain 16 of patient 6 by stimulator 4 may be delivered according to one or more programs executed by the stimulator and configured to efficaciously treat a neurological condition in the brain of the patient, e.g. epilepsy.

In one example, AAV9 is delivered to target site 24 within brain 16 of patient 6 via micropipette 22. The AAV vector transduces a nucleic acid encoding for ChR2 into axons of neurons whose cell bodies are near the distal ends of fibers 11A and 11B, and whose axons are near the original delivery site 24, though the distal ends of fibers 11A and 11B are remote from the original delivery site 24. Stimulator 4 generates and directs optical light 15 through fibers 11 to the neurons. Stimulator 4 generates optical light 15 with a wavelength and intensity that causes the ChR2 channel (being produced by the neuron from the nucleic acid with which it has been transduced) to become cation-permeable, activating the affected neurons within brain 16 of patient 6. Stimulator 4 may generate optical light 15 with a wavelength between about 420 nm and about 500 nm, such as between about 450 nm and about 495 nm, or in one example about 470 nm. Optical light 15 generated by stimulator 4 may, e.g., have an intensity of between about 0.5 mW/mm² and about 10 mW/mm², such as between about 1 mW/mm² and about 5 mW/mm², and in one example about 2.4 mW/mm². In one example, stimulator 4 may generate optical light 15 as a blue light with a wavelength of between about 450 nm and about 495 nm, such as between about 450 nm and about 470 nm.

In some examples, stimulator 4 may be configured to expose the target neurons to optical light 15 via fibers 11 for a pulse of between about 1 ms and about 1 second, such as between 5 ms and about 50 ms, and in one example about 10 ms. In one example, the ChR2 opsin may hold its activated state in the target neurons and slowly deactivate with a probability window of several seconds. In one example, stimulator 4 may be configured to deactivate or “switched off” the ChR2 opsin in the target neurons by optically stimulating the cells via fibers 11 with light characterized by a second wavelength and/or intensity. In one example, stimulator 4 may deactivate the ChR2 opsin in the target neurons within brain 16 of patient 6 by stimulating the neurons with a green light having a wavelength of between about 495 nm and about 570 nm, such as between about 510 nm and about 550 nm, and in one example about 535 nm. The green light generated by stimulator 4 in such an example may, e.g., have an intensity of between about 0.5 mW/mm² and about 10 mW/mm², such as between about 1 mW/mm² and about 5 mW/mm², and in one example about 2.4 mW/mm². In such examples, the ChR2 opsin may be exposed to the green light by stimulator 4 for a pulse of between about 20 ms and about 75 ms, such as between about 40 ms and about 60 ms, and in one example about 50 ms.

In one example, a viral vector with retrograde and/or anterograde transport properties, e.g. AAV9 is delivered to target site 24 and transduces a nucleic acid encoding for NpHR into the axons of neurons whose axons are near target site 24 and whose cell bodies are near the distal ends of fibers 11A and 11B, which are distal to the original delivery site 24. The nucleic acid transduced into the target neurons by the AAV9 may be in addition to or in lieu of the nucleic acid encoding for ChR2 described in the foregoing example. Stimulator 4 generates and directs optical light 15 through fibers 11 with a wavelength and intensity that causes the NpHR that is being expressed by the neurons as a result of their being transduced with the nucleic acid that encodes for NpHR to pump anions into the neuron lowering the resting potential of the neuron and inhibiting or deactivating the target neurons within brain 16 of patient 6. In one example, stimulator 4 may generate optical light 15 as a yellow light having a wavelength of between about 550 nm and about 610 nm, such as between about 570 nm and about 590 nm, and in one example about 580 nm. Yellow optical light 15 generated by stimulator 4 to control the NpHR opsin in the target neurons within brain 16 of patient 6 may, e.g., have an intensity of between about 0.5 mW/mm² and about 25 mW/mm², such as between about 10 and about 21 mW/mm² in one example or between about 1 mW/mm² and about 5 mW/mm² in another example. Stimulator 4 may be configured to expose the target neurons to optical light 15 via fibers 11 for between about 10 ms and about 1 second, such as between 20 ms and about 100 ms, and in one example about 40 ms.

In one example, stimulator 4 may be configured to optically stimulate the target neurons with optical light 15 via fibers 11 to activate the NpHR opsins in the cells in order to deactivate neurons that were previously activated via optical stimulation of, e.g., a ChR2 opsin as described above. In one example, a light pulse of a first wavelength, e.g. a 470 nm, is emitted by stimulator 4 via fibers 11 for about 10 ms to open ChR2 opsin channels to activate target neurons, which can remain active for several seconds. Stimulator 4 may then emit a light pulse of a second wavelength, e.g. 535 nm to close the ChR2 opsin channels. In another example, stimulator 4 may emit a light pulse with a 580 nm wavelength to activate the NpHR anion pump, thereby abruptly deactivating (preventing action potentials from firing in) the target neurons previously activated by optical opening of the ChR2 opsin channels.

Although the example of FIG. 1 employs individual optical fibers 11A and 11B to deliver light to two locations within brain 16 of patient 6, in other examples according to this disclosure, stimulator 4 may be connected to an array of optical electrodes, or optrodes that may deliver stimulation individually or together to multiple locations corresponding to each of the optrodes in the array. In one example, stimulator 4 may be connected to a thin sheet on which an array of optrodes is distributed. The sheet of optrodes may be configured to be implanted on the surface of brain 16 of patient 6, e.g. on the surface of the cerebral cortex under the dura mater.

Other means of light communication may be used in place of an optical fiber, including a wave guide, a hollow tube, a liquid filled tube, and a light pipe. In an alternative example, a light source, such as a light emitting diode (LED), is implanted at the target treatment site, e.g., at the distal end of a lead or on the housing of a microstimulator device implanted proximate target tissue, such that the light is emitted into the target tissue from the LED, rather than via an optical fiber. In this case, a conducting lead may be implanted to extend from an optical stimulation controller to the LED to conduct electrical energy to power the light source.

In the example of FIG. 1, optical fibers 11 may be made from a plastic or glass, and as such may provide advantages over the leads and electrodes used for in some examples of electrical stimulation. First, because optical fibers 11 are not electrically conducting in such examples, they do not provide a galvanic path for induced currents at the tissue interface so there is no risk of tissue capture or excessive heating that can occur due to modalities such as magnetic resonance imaging (MRI) or electromagnetic interference (EMI). Moreover, the elimination of conductors from the tissue interface helps to mitigate MRI interference that is seen with typical electrical stimulation electrodes, allowing for continued high-resolution imaging post-implant. Second, because there is not a relatively large electrical current flowing through the target tissue, as is the case with electrical stimulation, optical stimulation does not mask or block the relatively smaller bioelectric activity that is electrically sensed at the same time optical stimulation is delivered. Thus, optical stimulation allows, e.g. one or more of electrodes 17 on leads 12A and 12B to simultaneously electrical sense the resulting reaction by the target tissue, which, in turn, may allow system 2 to provide for closed-loop feedback and control of the optical stimulation.

As discussed above with respect to FIG. 1, system 2 may also include one or more sense electrodes 17 carried on one or more implantable leads 12A, 12B to permit implantable stimulator 4 to sense electrical signals from patient 6. Implantable leads 12A, 12B may be carried on lead body 10 and on each individual optical fiber 11. In this way, lead body 10 and optical fibers 11 act as leads for carrying sense electrodes 17. In another example, one or more optical fibers 11 and one or more conducting leads 12 may be carried together as a unitary lead that contains both the one or more optical fibers 11 and the conducting lead 12. In one example, a unitary lead may contain both an optical fiber and a conducting lead, wherein sense electrodes are placed on a lead sheath that covers the optical fiber. In this example, the electrical conductors could be axial, running along the length of the lead with the optical fiber extending alongside the electrical conductor, or with the optical fiber being wound in a coil around the one or more electrical conductors, or with the electrical conductor being embedded within the optical fiber. In another example, the one or more electrical conductors may be wound in a coil, with the optical fiber extending inside the center of the coil, or with the optical fiber wound in a generally coaxial coil. In another example, rather than optical fibers, system 2 may include a lead that carries one or more electrical conductors that provide power to one or more light sources, such as an LED or a laser, located at the distal end of the lead. The lead may also carry one or more sense electrodes that are coupled to one or more of the electrical conductors carried by the lead. The conductors coupled to a light source or to a sense electrode may be arranged in any of the configurations described above with respect to electrical conductors or optical fibers above.

The sense electrodes 17 of lead segments 12A, 12B are shown as ring electrodes. Ring electrodes are commonly used in DBS applications because they are simple to program and are capable of sensing an electrical field to any tissue proximate to lead segments 12A, 12B. In other implementations, sense electrodes 17 of lead segments 12A, 12B may have different configurations. For example, the electrodes of lead segments 12A, 12B may have a complex electrode array geometry that is capable of sensing bioelectric potentials in a directional or localized manner.

FIG. 1 further depicts a housing electrode 13 that may be used in conjunction with or in place of sense electrodes 17. In some cases, housing 14 may include multiple housing electrodes. Housing electrode 13 may be formed integrally with an outer surface of hermetically-sealed housing 14 of implantable stimulator 4, also referred to in this disclosure as implantable medical device (IMD) 4, or otherwise coupled to housing 14. Housing electrode 13 may be used to form unipolar electrode combinations with one or more electrodes carried on leads 12A, 12B to sense bioelectric potentials. Alternatively, electrodes carried on leads 12A, 12B may be used in bipolar or multipolar combinations to sense bioelectric potentials. To sense bioelectric potentials in proximity to the tissue illuminated by the optical stimulation, at least one of the electrodes in a given electrode combination should be positioned near the distal end of an optical fiber 11A, 11B.

A proximal end of lead body 10 may be both optically and mechanically coupled to header 8 on implantable stimulator 4 either directly or indirectly via an optical extension. Alternatively, lead body 10 may be optically and mechanically coupled to a window as described above. Optical fibers 11 permit passage of light energy along the body of optical fibers 11 to connect the distal ends of fibers 11 to a light source in implantable stimulator 4. Lead body 10 traverses from the implant site of implantable stimulator 4 along the neck of patient 6 to cranium 18 of patient 6 to access brain 16. Optical fibers 11A and 11B may be implanted within the right and left hemispheres, respectively, in order to deliver optical stimulation to one or more regions of brain 16, which may be selected based on the patient condition or disorder. Alternatively, a single optical fiber 11 may be implanted at a specific treatment point within brain 16, e.g. proximate the cerebral cortex or the hippocampus, or multiple optical fibers 11A, 11B may each be directed at the specific treatment target site, wherein the treatment target site may be selected based on the patient condition or disorder.

Therapy system 2 also may include programmer 20, which may be configured as a clinician and/or a patient programmer. In some examples, system 2 may include both a clinician and a separate patient programmer. Programmer 20 may be a handheld computing device that permits a clinician to program stimulation therapy for patient 6 via a user interface, e.g., using input keys and a display. Programmer 20 may also include a display and input keys to allow patient 6 to interact with the programmer and implantable stimulator 4. In one example, a clinician employing programmer 20 may specify stimulation parameters, i.e., create programs, for use in delivery of stimulation therapy. In another example, patient 6 may employ programmer 20 to start, stop or adjust optical stimulation therapy. For example, programmer 20 may permit patient 6 to adjust stimulation parameters of a program such as duration of treatment, optical intensity or amplitude, pulse width, pulse frequency, burst length, and burst rate. Patient 6 may also select a program, e.g., from among a plurality of stored programs, as the present program to control delivery of stimulation by implantable stimulator 4. Programmer 20 may support telemetry (e.g., radio frequency (RF) telemetry) with implantable stimulator 4 to download programs and, optionally, upload operational or physiological data stored by implantable stimulator 4. In this manner, e.g., a clinician may periodically interrogate implantable stimulator 4 to evaluate efficacy and, if necessary, modify the programs or create new programs. In some examples, programmer 20 may be configured as a clinician programmer and may transmit programs to another programmer, e.g. a patient programmer in addition to or instead of implantable stimulator 4.

In some examples, implantable stimulator 4 delivers stimulation according to a group of programs at a given time. Each program of such a program group may include respective values for each of a plurality of therapy parameters, such as respective values for each of optical intensity or amplitude, pulse width, pulse shape, pulse rate, burst frequency, burst rate, burst width, and optical fiber configuration (e.g., the combination of optical fibers used and with what light intensity and wavelengths). Implantable stimulator 4 may interleave pulses or other signals according to the different programs of a program group, e.g., cycle through the programs, to simultaneously treat different symptoms or different body regions, or provide a combined therapeutic effect. In such examples, programmer 20 may be used by a clinician to create programs, and assemble the programs into program groups. Additionally, programmer 20 may be used by patient 6 to adjust stimulation parameters of one or more programs of a program group, and select a program group, e.g., from among a plurality of stored program groups, as the current program group to control delivery of stimulation by implantable stimulator 4.

Implantable stimulator 4 and programmer 20 may communicate via cables or a wireless communication, as shown in FIG. 1. Programmer 20 may, for example, communicate via wireless communication with implantable stimulator 4 using RF telemetry techniques. Programmer 20 may communicate using any of a variety of local wireless communication techniques, such as RF communication according to the 802.11 or Bluetooth specification sets, infrared communication, e.g., according to the IrDA standard, or other standard or proprietary telemetry protocols. Programmer 20 may include a transceiver to permit bi-directional communication with implantable stimulator 4 and/or other devices.

In the example of FIG. 1, implantable stimulator 4 may deliver, for example, deep brain stimulation (DBS) or cortical stimulation (CS) therapy to patient 6 via the optical fibers 11 to treat any of a variety of neurological disorders or diseases. Example neurological disorders may include depression, dementia, obsessive-compulsive disorder and movement disorders, such as Parkinson's disease, spasticity, epilepsy, and dystonia. DBS also may be useful for treating other patient conditions, such as migraines and obesity. However, this disclosure is not limited to the configuration of lead body 10 or optical fibers 11 shown in FIG. 1, or to the delivery of DBS or CS therapy.

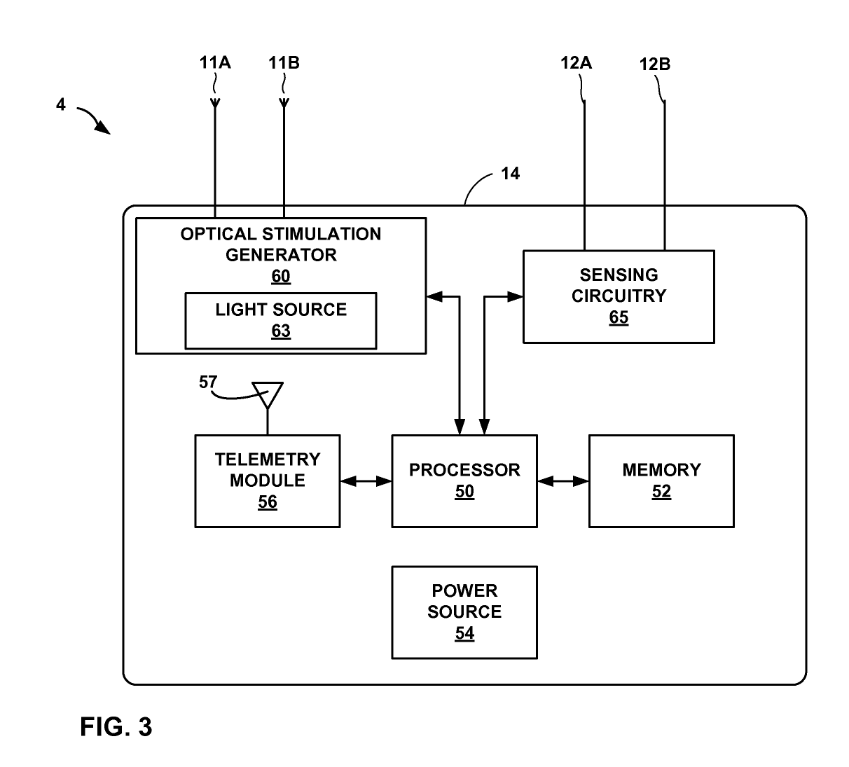

FIG. 3 is a block diagram illustrating various components of an example of implantable stimulator 4 of FIG. 1. Example configuration of stimulator 4 of FIG. 3 includes processor 50, memory 52, power source 54, telemetry module 56, antenna 57, optical stimulation generator 60, and sensing circuitry 65. Optical stimulation generator 60 includes light source 63, which is coupled to first and second optical fibers 11A and 11B, respectively. Sensing circuitry is coupled to first and second electrical leads 12A and 12B, respectively.

Implantable stimulator 4 may be a multi-channel device in the sense that it may be configured to include multiple optical paths (e.g., multiple light sources and optical fibers) that may deliver different optical stimulation waveforms, some of which may have different wavelengths. Although two optical fibers are shown in FIG. 3, more or less optical fibers may be used in different implementations, such as one, two, five or more optical fibers and associated light sources may be provided. The optical fibers may be detachable from housing 14 of implantable stimulator 4, or be fixed to the housing.

In one example of stimulator 4, multiple optical fibers may be provided to a single target tissue site within patient 6 in the form of one or more optical fiber bundles that may be the same or different from fiber bundle to fiber bundle. In another example, a set of one or more optical fibers or fiber bundles may be provided from stimulator 4 to a first target tissue site within patient 6 and a second set of one or more optical fibers or fiber bundles may be provided from the stimulator to a second target tissue site. For example, a set of one or more optical fibers or fiber bundles may be directed from stimulator 4 to the subthalmic nucleus (STN) of patient 6 and another set of one or more optical fibers or fiber bundles may be directed to the pedunculopontine nucleus such that the combined use of the two sets of optical fibers by stimulator 4 may provide closed-loop DBS to treat movement disorders such as Parkinson's disease, spasticity, epilepsy, and dystonia. In another example, two or more sets of optical fibers or optical fiber bundles may be placed at various epileptic foci and used for distributed treatment of the epileptic foci. In some examples, one or more optical fibers or fiber bundles may be selectively turned on or off in order to manage power.

Memory 52 may store instructions for execution by processor 50, optical stimulation therapy data, sensor data, and/or other information regarding therapy for patient 6. Processor 50 may control optical stimulation generator 60 to deliver stimulation according to a selected one or more of a plurality of programs or program groups stored in memory 52. Memory 52 may include any electronic data storage media, such as random access memory (RAM), read-only memory (ROM), electronically-erasable programmable ROM (EEPROM), flash memory, or the like. Memory 52 may store program instructions that, when executed by processor 50, cause the processor to perform various functions ascribed to processor 50 and implantable stimulator 4 in this disclosure.

Information stored on the memory 52 may include information regarding therapy that the patient 6 had previously received. Storing such information may be useful for subsequent treatments such that, for example, a clinician may retrieve the stored information to determine the therapy applied to the patient during his/her last visit, in accordance with this disclosure.

Processor 50 may include one or more microprocessors, digital signal processors (DSPs), application-specific integrated circuits (ASICs), field-programmable gate arrays (FPGAs), or other digital logic circuitry. Processor 50 controls operation of implantable stimulator 4, e.g., controls stimulation generator 60 to deliver stimulation therapy according to a selected program or group of programs retrieved from memory 52. For example, processor 50 may control stimulation generator 60 to deliver optical signals, e.g., as stimulation pulses, with intensities, wavelengths, pulse widths (if applicable), and rates specified by one or more stimulation programs. Processor 50 may also control optical stimulation generator 60 to selectively deliver the stimulation via optical fibers 11A and 11B in different optical fiber combinations, and with stimulation specified by one or more programs.

Upon selection of a particular program group, processor 50 may control optical stimulation generator 60 to deliver optical stimulation according to the programs in the groups, e.g., simultaneously or on a time-interleaved basis. A group may include a single program or multiple programs. As mentioned previously, each program may specify a set of stimulation parameters, such as amplitude, pulse width and pulse rate, if applicable. In addition, each program may specify a particular optical fiber combination for delivery of optical stimulation. The optical fiber combination may specify particular optical fibers in a single array or multiple arrays.

Optical stimulation generator 60 is optically coupled to optical fibers 11A and 11B. As noted above, in another example optical stimulation generator may be coupled to more or less than two optical fibers 11. Optical stimulation generator 60 may include stimulation generation circuitry to generate stimulation pulses and circuitry for switching stimulation across different optical fiber combinations, e.g., in response to control by processor 50. Optical stimulation generator 60 produces an optical stimulation signal in accordance with a program based on control signals from processor 50. Optical stimulation generator 60 may also include one or more light sources 63, such as one or more lasers or one or more light-emitting diodes (LEDs) that produce optical light within stimulator 4 that is then transmitted along optical fibers 11 to provide optical stimulation treatment to a target tissue. Alternatively, light source 63 may be separate from optical stimulation generator 60 such that optical stimulation generator 60 provides the signal that powers light source 63.

As described above, in one example, stimulator 4 delivers optical stimulation to target neurons within brain 16 of patient 6 that have been transduced with one or more opsin proteins. The optical stimulation delivered by stimulator 4 may be configured to activate the light-sensitive proteins in order to modulate the behavior of the target neurons, e.g. to modulate the action potential to activate and/or inactivate the neuron cells. In one example, as described above, a first opsin is activated by a first wavelength of light delivered by stimulator 4 via optical stimulation generator 60, light source 63 and fibers 11 so that the target neurons become permeable to cations to initiate neuronal spikes and fire the target neuron and a second opsin is activated by a second wavelength of light to deactivate or inhibit the target neurons. In this example, stimulator 4 may provide for a useful chronic device enabling continuous therapy by providing light at both the first wavelength and the second wavelength on an alternating or selective basis in order to provide for both selective activation and inhibition of the target neurons.

For example, if ChR2 is used as the activation opsin and NpHR is used as the inhibition opsin, then optical stimulation generator 60, and particularly light source 63, may be controlled by processor 50 to provide light of a first wavelength of between about 420 nm and about 475 nm, such as about 450 nm, to activate the ChR2 and activate the target neurons and a second wavelength of between about 510 nm and about 580 nm, such as about 535 nm, to activate the NpHR and inhibit the target neurons. In another example, optical stimulation generator 60 may be configured to stimulate target neurons within brain 16 of patient 6 to open a ChR2 channel via brief pulses of light with a wavelength of about 450 nm with an intensity of between about 8 mW/mm² and about 12 mW/mm² and a duty cycle of about 1% and a pulse frequency of between about 100 Hz and about 120 Hz, such as a 100 μs pulse every 10 ms. In one example, an NpHR opsin is activated, thus deactivating the target neuron, when exposed to yellow light from light source 63 via fibers 11 by optical stimulation generator 60 having a wavelength of between about 550 nm and about 610 nm, such as between about 570 nm and about 590 nm, and in one example about 580 nm.

In some examples, because light transmitted from light source 63 to tissue within brain 16 of patient 6 may scatter and because the required voxel size for therapy may not be well established, optical stimulation generator 60 may be designed to be scaleable with the ability to incorporate multiple stimulation circuits and outputs to increase the volume of activation by using multiple pathways. In another example, one or more lenses may be used to focus or dissipate the light, as desired to provide more intense or scattered optical stimulation in the target tissue. In one example, a lens may be created at distal ends optical fibers 11 by modifying or distorting the distal end of each fiber to form a lens that may focus or dissipate light once it reaches the distal end.

Optical stimulation generator 60 may provide for the two wavelengths of stimulation light by having two light sources, one for each wavelength, such as a LED dedicated to each wavelength of light that feed into optical fibers 11 to deliver each wavelength of light to the target neurons depending on which LED is activated by optical stimulation generator 60 as controlled by processor 50. Alternatively, a single light source 63 that is capable of emitting both wavelengths may be used, such as a tunable LED or other tunable light source, wherein a particular wavelength is selected based on a treatment program run by processor 50 that causes optical stimulation generator 60 to control light source 63 to emit the selected wavelength, such as by tuning the tunable LED or other tunable light source to the selected wavelength.

Optical stimulation generator 60 may also control, under the direction of processor 50, several other parameters with respect to the optical stimulation of the target tissue, such as the intensity of light emitted to stimulate the target tissue, the number of pulses of light to be emitted, the pulse width, the frequency of pulses, and the pattern of pulses, including burst patterns wherein optical stimulation generator 60 may control burst width, burst frequency, the number of pulses per burst, and the number of bursts. In one example, optical stimulation generator 60 is capable of delivering light from light source 63 via one or more of optical fibers 11 with an intensity of between about 1 mW/mm² and about 5 mW/mm². When light pulses are used as part of a stimulation program, optical stimulation generator 60 may, for example, produce pulses with a pulse width of between about 100 μs and about 15 ms, such as pulses with a pulse width of about 10 ms, and with a pulse frequency of between about 0.1 Hz and about 1 kHz, such as a frequency of about 0.2 Hz. In one example, optical stimulation generator 60 may drive optical stimulation with programmable modulation patterns that mimic existing patterns used in electrical stimulation for deep brain stimulation (DBS), and also allow for novel patterns that leverage the capabilities of the inhibitory optical transducer in the cell membranes.

Additional information regarding electrical circuitry that may comprise examples of optical stimulation generator 60 are described in U.S. application Ser. No. 12/951,766, filed Nov. 22, 2010, which claims the benefit of U.S. Provisional Application No. 61/264,550, filed Nov. 25, 2009 and of U.S. Provisional Application No. 61/301,836, filed Feb. 5, 2010, the entire contents of all of which are incorporated herein by this reference.

Referring again to FIG. 3, telemetry module 56 may include a radio frequency (RF) transceiver to permit bi-directional communication between implantable stimulator 4 and programmer 20. Telemetry module 56 may include an antenna 57 that may take on a variety of forms. For example, antenna 57 may be formed by a conductive coil or wire embedded in a housing associated with medical device 4. Alternatively, antenna 57 may be mounted on a circuit board carrying other components of implantable stimulator 4 or take the form of a circuit trace on the circuit board. In this way, telemetry module 56 may permit communication with programmer 20 in FIG. 1 or another peripheral device communicatively coupled with stimulator 4, to receive, for example, new programs or program groups, or adjustments to programs or program groups. Telemetry module 56 may also permit communication with programmer 20 to receive, for example, an image captured by the programmer of the lead placement along with information regarding the captured image and the therapy received by the patient during previous sessions, in accordance with this disclosure. Telemetry module 56 may also communicate information regarding previous therapy sessions that have been stored in memory 52, to an external programmer during a subsequent therapy session; the information regarding a previous therapy session may have been imported by a programmer used in the previous session.

Power source 54 may be a non-rechargeable primary cell battery or a rechargeable battery and may be coupled to power circuitry. However, the disclosure is not limited to examples in which the power source is a battery. In another example, power source 54 may comprise a supercapacitor. In some examples, power source 54 may be rechargeable via induction or ultrasonic energy transmission, and include an appropriate circuit for recovering transcutaneously received energy. For example, power source 54 may be coupled to a secondary coil and a rectifier circuit for inductive energy transfer. In additional examples, power source 54 may include a small rechargeable circuit and a power generation circuit to produce the operating power. Recharging may be accomplished through proximal inductive interaction between an external charger and an inductive charging coil within stimulator 4. In some examples, power requirements may be small enough to allow stimulator 4 to utilize patient motion at least in part and implement a kinetic energy-scavenging device to trickle charge a rechargeable battery. A voltage regulator may generate one or more regulated voltages using the battery power.

In one example, stimulator 4 may also include a temperature sensor to monitor the temperature at stimulator 4 or proximate to stimulator 4 during optical stimulation and recharge. Such a temperature sensor may be used to adjust light delivery to target tissue within brain 16, or another location within patient 6 based on the temperature sensed by the sensor. For example, the temperature sensor, in conjunction with processor 50, may be used to ensure that the peak temperature at the stimulation site is constrained to under a 2° C. increase over nominal body temperature per regulator (e.g., Food and Drug Administration (FDA)) guidelines, which can be a concern for light that is needed for driving less efficient opsin channels. In one example, if the temperature sensor determines a rise in temperature above a permitted temperature, such as more than about a 2° C. (degrees Celsius) increase over nominal body temperature, processor 50 may modulate power in the device to avoid overheating of tissue. In another example, processor 50 may cease optical stimulation and, in some examples, switch to electrical stimulation to mitigate the risk of overheating. In another example, adjusting the delivery of light from light source 63 based on sensor input, e.g. from the temperature sensor may include adjusting one or more of pulse rate, pulse width, amplitude intensity, or duty cycle of light delivered from light source 63.

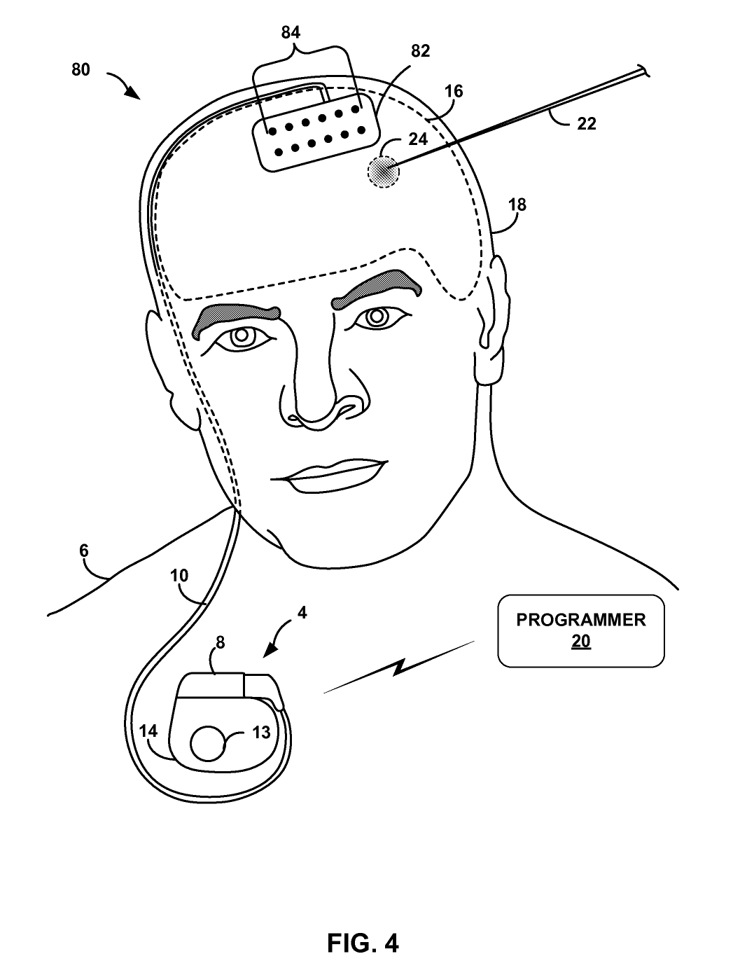

FIG. 4 is a conceptual diagram illustrating example system 80 that may be used to deliver stimulation therapy to patient 6. Therapy system 80 includes implantable stimulator 4, lead body 10, and programmer 20, which may have the same general configurations and functions as described above with reference to system 2 of FIG. 1. In the example of FIG. 4, however, stimulator 4 is connected to optrode array 82 via lead body 10, instead of being connected to individual optical fibers, such as fibers 11A and 11B in the example of FIG. 1. Optrode array 82 includes a number of optical electrodes, e.g. optrodes 84. The specific number of optrodes in optrode array 82 may vary in different examples according to this disclosure. Additionally, the specific configuration of optrode array 82, e.g. size and shape, may vary in different examples of therapy systems according to this disclosure.

In one example, optrode array 82 includes a thin sheet or paddle type medical lead body on one more surfaces of which optrodes 84 are arranged. For example, in FIG. 4, optrode array 82 is includes a generally rectangular shaped body characterized by major rectangular shaped surfaces on which optrodes 84 are arranged. In one example, optrode array 82 may be fabricated from a flexible biocompatible material such that it may flex to contours within brain 16 of patient 6. For example, optrode array 82 may include a thin sheet fabricated from a flexible material such that the array is configured to be implanted on the surface of the cerebral cortex of brain 16 of patient 6 under the dura mater. In another example, optrode array 82 may be fabricated from a rigid material designed to retain a preconfigured shape after implantation.

One or more optical fibers may be bundled in lead body 10 to optically connect optrodes 84 to stimulator 4, and, in particular to a light source within or associated with the stimulator. The optical connection between stimulator 4 and optrodes 84 on array 82 may be configured such that the optrodes are capable of delivering stimulation individually or together, as a whole or in different sets, e.g. different columns or rows of optrodes, to multiple locations of brain 16 corresponding to each of the optrodes in the array. In one example, one optical fiber may be carried by lead body 10 to optrode array 82 to drive all of optrodes 84 to deliver optical stimulation to brain 16. In another example, a number of optical fibers may be bundled in lead body 10 and each fiber may be coupled to a respective individual optrode 84 or one of several sets of optrodes 84 to deliver stimulation at individual sites or regions, respectively, within brain 16 of patient 6. In examples in which one fiber delivers light to multiple optrodes 84 of optrode array 82, an optical splitter, e.g. a beam splitter may be employed to divide the light from stimulator 4. In one example, the divided beam of light may be delivered through array 82 to each optrode 84 via respective optical fibers included in the array. Additionally, in one example, each optrode 84 may include a lens to direct light from stimulator 4 through one or more optical fibers to cells within patient 6.

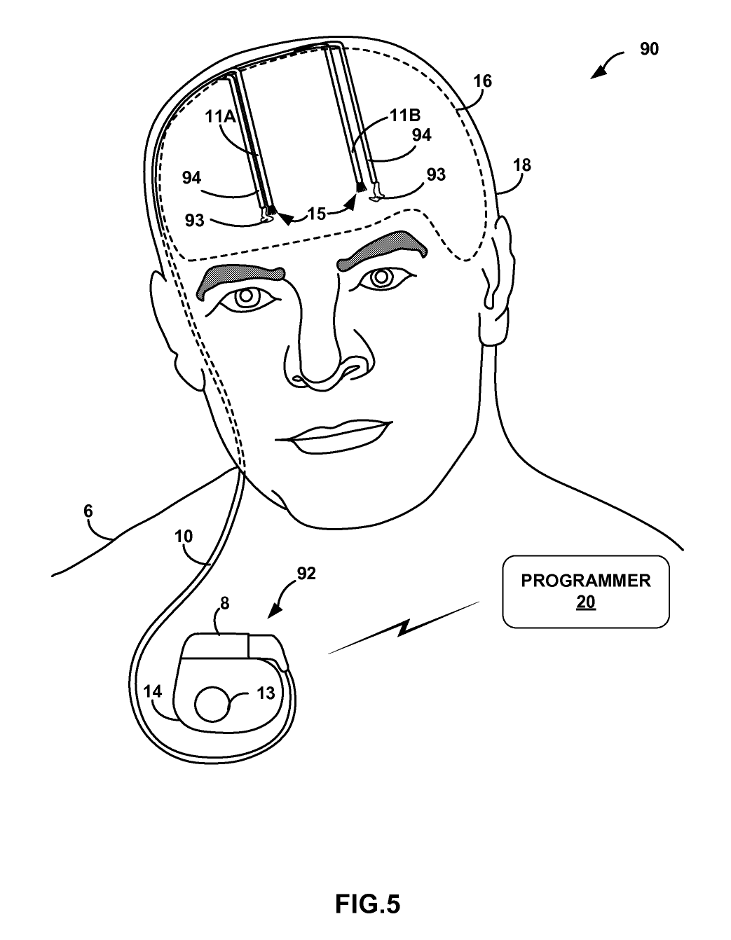

A conceptual diagram illustrating another example therapy system 90 is shown in FIG. 5. Like systems 2 and 30 described above, therapy system 90 is used to deliver optical stimulation therapy to patient 6. Therapy system 90 includes an implantable medical device (IMD) 92 that delivers optical stimulation to patient 6 via one or more implantable optical fibers 11. With respect to functions related to optical stimulation provided by IMD 92, the device in system 90 may be configured and function in essentially the same as described above with reference to stimulator 4 in FIG. 1. In system 90, however, IMD 92 also provides the ability to deliver a therapeutic agent 93 to a target site within patient 6.

An example therapeutic agent that IMD 92 may be configured to deliver is a gene therapy agent that provides targeted delivery of a light-sensitive ion channel protein, also referred to as an “opsin,” to specific target cells, such as neurons within brain 16 or the spinal cord of patient 6, such as by contacting the target cells with a vector, such as a viral vector with known retrograde and/or anterograde transport properties and including a nucleic acid sequence that codes for the opsin. For example, IMD 92 may deliver an AAV vector capable of transduction of a genetic agent encoding for an opsin not only into cells at a delivery site within brain 16 of patient 6 at which the vector is initially delivered, but also to cells in a plurality of regions within the patient proximal and remote to the delivery site.

In other examples, IMD 92 may be configured to deliver other therapeutic agents to patient 6 in various locations with the patient's body and to treat various conditions. For example, IMD 92 may be configured to deliver a therapeutic agent to patient 6 that treats a condition independent of optical stimulation delivered by the device. In such an example, the viral vector employed to transduce cells within patient 6 with an opsin that may be activated via optical stimulation by IMD 92 may be delivered to patient 6 not from the IMD, but, instead, e.g. via intravenous or intracranial injection of the vector into the patient. In one example, IMD 92 may be configured to deliver the viral vector to a delivery site within patient 6 in addition to or in lieu of other therapeutic agents the device delivers to the patient.

In one example, IMD 92 delivers a therapeutic fluid to patient 6 through one or more catheters 94 coupled to IMD 92 that are implanted so that a distal end of catheter 94 is located proximate to the target cells. Stereotactic techniques or other positioning techniques may be used to precisely position fluid delivery catheters and/or optical fibers with respect to target tissue sites and to maintain the precise positioning throughout use. In some examples, after positioning, one or more fluid delivery catheters and/or optical fibers may be held precisely in place using fixation techniques or mechanisms such as those similar to the Medtronic StimLoc™ burr hole cover, manufactured by Medtronic, Inc., of Minneapolis, Minn. Additionally, in some examples, an optical stimulation generator, i.e., including a light source, controller, power source, and telemetry circuitry, instead of included in IMD 92 as described with reference to stimulator 4 in FIG. 1, may formed as a microstimulator that is structurally mounted on or integrated with a burr hole cover, such as a StimLoc™ burr hole cover. In this case, the optical fiber would run only from the skull to deep brain structures, instead of running from an implant pocket, such as an implant pocket near the clavicle. For this example, in some cases, the microstimulator device can be anchored to the skull using one or more bone morphogenetic proteins (BMPs), which is a material used in spine and biologics procedures to fuse cervical or spinal discs.

In one example, one or more catheters 94 are provided to deliver the therapeutic agent 93 at or near the same location within the target tissue that is exposed to light 15 so that the therapeutic agent will promote expression of the opsin protein by the target tissue at the same point where the target tissue will be exposed to optical stimulation. Catheters 94 could be side by side with optical fibers 11, as shown in FIG. 5, or the optical fibers and fluid delivery conduits may be combined into a common unitary lead, such as within separate lumens within the unitary lead with a fluid conduit in side-by-side arrangement with an optical fiber or in a coaxial arrangement with the optical fiber being within the fluid conduit or vice versa. In one example (not shown), the optical fiber that delivers light stimulation to the target tissue may be a fiber with a hollow core so that light is passed through the outer fiber portion while the therapeutic agent is passed through the hollow core. In this case, the fiber may have an annular cross-section. In yet another example, a conduit may be provided that delivers the therapeutic agent to the target tissue while the optical fiber is threaded through the conduit such that the therapeutic agent is delivered in the annular region between the outer diameter of the optical fiber and the inner wall of the lead body.

Catheter 94 can comprise a unitary catheter or a plurality of catheter segments connected together to form an overall catheter length. An external programmer, such as programmer 20 may be configured to wirelessly communicate with IMD 92 as needed, such as to provide or retrieve therapy information or control aspects of therapy delivery (e.g., modify the therapy parameters such as rate or timing of delivery, turn IMD 92 on or off, and so forth) from IMD 92 to patient 6.

In the example of FIG. 5, IMD 92 delivers a therapeutic agent through catheter 94 to target sites within brain 16. In other examples, IMD 92 may be configured for intrathecal delivery into the intrathecal space, as well as epidural delivery into the epidural space, both of which surround the spinal cord. In some examples, the target delivery site in other applications of therapy system 90 can be located within patient 6 proximate to, e.g., sacral nerves (e.g., the S2, S3, or S4 sacral nerves), the spinal cord, or any other suitable nerve, organ, muscle or muscle group in patient 6, which may be selected based on, for example, a patient condition. In one such application, therapy system 90 may be used to deliver a therapeutic agent to tissue proximate to a pudendal nerve, a perineal nerve or other areas of the nervous system, in which cases, catheter 94 would be implanted and substantially fixed proximate to the respective nerve.

In one example, IMD 92 can deliver one or more therapeutic agents to patient 6 according to one or more dosing programs that set forth different therapy parameters, such as a therapy schedule specifying programmed doses, dose rates for the programmed doses, and specific times to deliver the programmed doses. The dosing programs may be a part of a program group for therapy, where the group includes a plurality of dosing programs and/or therapy schedules. In some examples, IMD 92 may be configured to deliver a therapeutic agent to patient 6 according to different therapy schedules on a selective basis. IMD 92 may include a memory to store one or more therapy programs defining therapy deliverd to patient 6, as well as instructions defining the extent to which patient 6 may adjust therapy parameters, switch between dosing programs, or undertake other therapy adjustments. Such programs and other instructions stored on memory of IMD 92 may be executed by one or more processors included in the device or by a processor of programmer 20 or another device communicatively connected to IMD 92.

IMD 92 may include one or more reservoirs in which one or more therapeutic fluids are stored and a pumping mechanism configured to draw fluid from the reservoir and deliver it to target sites within patient 6, e.g. sites within brain 16 of the patient as illustrated in FIG. 5. In one example, during operation of IMD 92, a processor of the device may control a fluid delivery pump with the aid of instructions associated with program information that is stored in memory to deliver a therapeutic fluid to patient 16 via catheter 94. Pumping mechanisms included in IMD 92 may be any mechanism that delivers a therapeutic fluid in some metered or other desired flow dosage to the therapy site within patient 6 from the reservoir(s) of the IMD via catheter 94. In one example, IMD 92 includes a squeeze pump that squeezes internal tubing within the IMD in a controlled manner, e.g., such as a peristaltic pump, to progressively move fluid from a reservoir to the distal end of catheter 94 and then into patient 6 according to parameters specified by a therapy program stored on memory and executed by a processor. In various other examples, IMD 92 may include an axial pump, a centrifugal pump, a pusher plate pump, a piston-driven pump, or other means for moving fluid from a reservoir and through catheter 94 to patient 6. In one example, IMD 92 may include an electromechanical pump that delivers fluid by the application of pressure generated by a piston that moves in the presence of a varying magnetic field and that is configured to draw fluid from a reservoir and pump the fluid through catheter 94 to patient 6.

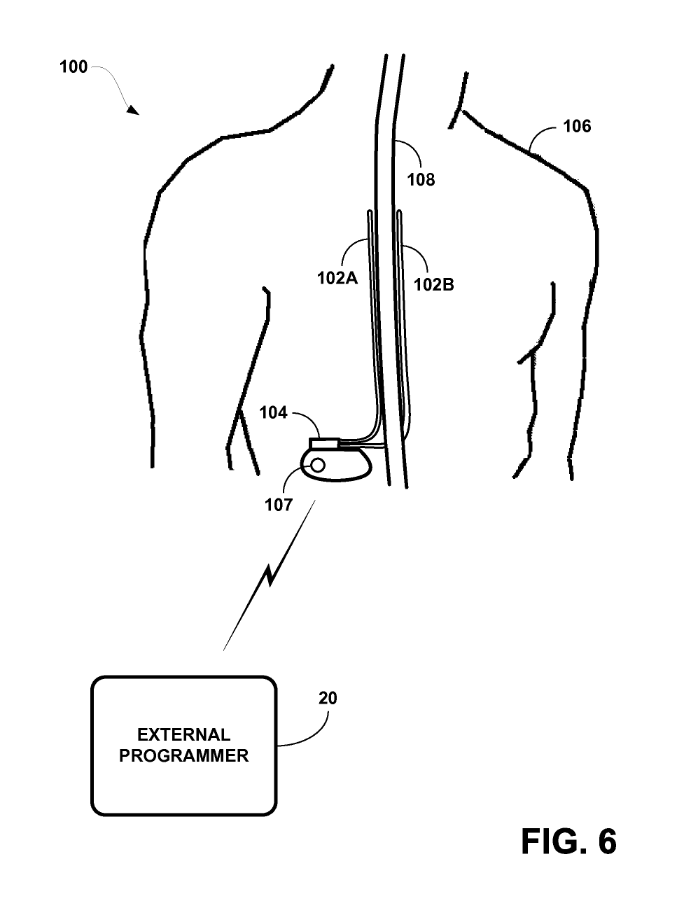

FIG. 6 is a conceptual diagram illustrating system 100 that delivers stimulation therapy to spinal cord 108 of patient 106, also known as spinal cord stimulation (SCS). Other stimulation systems may be configured to deliver stimulation to gastrointestinal organs, pelvic nerves or muscle, peripheral nerves, or other stimulation sites. In the example of FIG. 6, system 100 delivers optical stimulation therapy from implantable stimulator 104 to spinal cord 108 via one or more optical fibers 102A and 102B (collectively “optical fibers 102”). System 100 and, more particularly, implantable stimulator 104 may operate in a manner similar to implantable stimulator 4 of FIG. 1. That is, in one example, implantable stimulator 104 delivers controlled optical stimulation pulses or waveforms to patient 106 via one or more regulated stimulation optical fibers 102.

In the example of FIG. 6, the distal ends of optical fibers 102 are placed adjacent to the target tissue of spinal cord 108 such that light is emitted from the distal ends into the target tissue. The proximal ends of optical fibers 102 may be both optically and mechanically coupled to implantable stimulator 104 either directly or indirectly via a fiber extension and header. Alternatively, in some examples, optical fibers 102 may be implanted and coupled to an external stimulator, e.g., through a percutaneous port.

Stimulator 104 may be implanted in patient 106 at a location minimally noticeable to the patient. For SCS, stimulator 104 may be located in the lower abdomen, lower back, buttock or other location to secure the stimulator. Optical fibers 102 are tunneled from stimulator 104 through tissue to reach the target tissue adjacent to spinal cord 108 for optical stimulation delivery. Light is directed through optical fibers 102 so that the light is emitted from the distal ends of leads 102 in order to provide optical stimulation pulses from each optical fiber 102 to the target tissue. In one example, optical fibers 102 are anchored within or along the spinal column to prevent migration of optical fibers 102 after implantation. Anchoring of optical fibers 102 may prevent migration of optical fibers 102 during use and may also allow optical fibers 102 to be subjected to controlled bends to prevent light leakage from the optical fibers 102. In another example, optical fibers are not used, but rather the light source is implanted at the target tissue at the end of an electrode, such as implanting a wire that extends from the stimulator 104 to the target tissue to power an LED to expose the target tissue to light. In another example, an LED is carried on the housing of a device that is implanted proximate the target tissue to expose the target tissue to light, while the device, such as a microstimulator device, may be connected to an implantable stimulator that is connected to the device by a wire. In still another example, an LED within the device housing may deliver light through a window in the device to target tissue proximate the window.

Implantable stimulator 104 may deliver stimulation to spinal cord 108 to reduce the amount of pain perceived by patient 106. As mentioned above, however, the stimulator may be used with a variety of different therapies, such as peripheral nerve stimulation (PNS), peripheral nerve field stimulation (PNFS), pelvic floor stimulation, gastric stimulation, and the like. The stimulation delivered by implantable stimulator 104 may take the form of optical stimulation pulses or bursts, and may be characterized by controlled light intensity, as well as programmed pulse widths and pulse rates in the case of stimulation current pulses or controlled burst widths, burst frequencies, and burst rates for optical stimulation bursts.

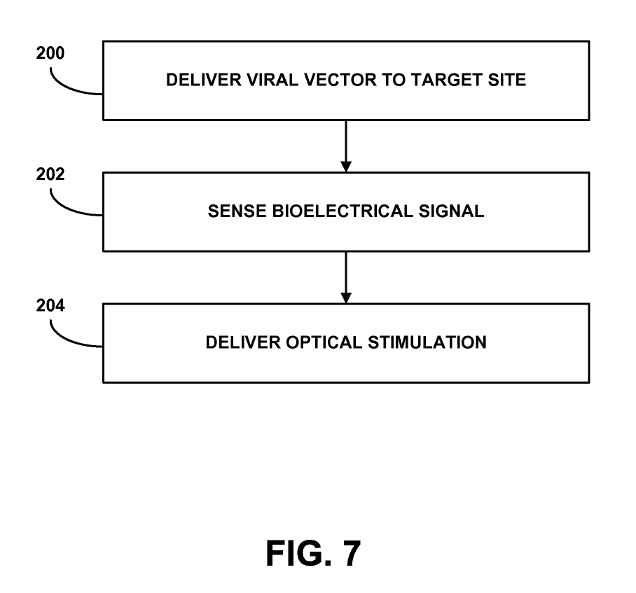

FIG. 7 is a flow chart illustrating a method of optogenetically modulating a target population of cells within a patient. The example method of FIG. 7 includes delivering a viral vector comprising a genetic agent encoding for one or more light-sensitive proteins to a target site within a patient (200), sensing a bioelectrical signal related to a neurological condition of the patient (202), and delivering optical stimulation to one or more cells transduced with the genetic agent by the viral vector to treat the neurological condition of the patient (204).

The functions of the method of FIG. 7 for optogenetically modulating a target population of cells within a patient are described as carried out by various components of therapy system 2 of FIG. 1. However, in other examples, one or more of these functions may be carried out by other devices including, e.g., devices associated with the systems described with reference to FIGS. 4-6. For example, instead of delivering optical stimulation to patient 6 in methods according to the example of FIG. 7 via optical fibers 11, as illustrated in FIG. 1, in one example, stimulator 4 may deliver optical stimulation to tissue within brain 16 of the patient via optrodes 84 arranged on optrode array 82.

Referring again to FIG. 7, the example method includes delivering a viral vector comprising a genetic agent encoding for one or more light-sensitive proteins to a target site within a patient (200). In one example, the method of FIG. 7 includes delivering a viral vector comprising a genetic agent encoding for light-sensitive proteins, i.e. an opsin, to a single region of brain 16 of patient 6, in which the viral vector delivering the genetic agent is configured to transduce the agent into cells in a number of regions of the brain proximal and remote to the delivery region. As noted above with reference to FIG. 1, while a number of types of viral vectors are capable of transducing cells with a nucleic acid encoding for an opsin, examples according to this disclosure employ viral vectors with retrograde and/or anterograde transport properties. In particular, examples according to this disclosure employ viral vectors that are capable of retrograde and/or anterograde transport after initial delivery within patient 6 to sites other than the delivery site such that the nucleic acid encoded for the opsin is transduced to cells not only at the delivery site, but also to cells in a plurality of regions within the patient proximal and remote to the delivery site.

A number of viral vectors with retrograde and/or anterograde transport properties may be employed in examples according to this disclosure. In one example, an AAV vector is capable of transduction of a genetic agent encoding for an opsin into cells not only at a delivery site within patient 6 at which the vector is initially delivered, but also to cells in a plurality of regions within the patient proximal and remote to the delivery site. Different types of AAV vectors may be employed in examples according to this disclosure. In some examples, AAV vectors may include single-stranded DNA (rAAV) that requires host-cell synthesis of the complementary DNA strand for transduction. However, in other examples, an AAV vector may include double-stranded DNA, or dimeric inverted repeat DNA molecules. Dimeric, or self-complementary (scAAV) may be capable of spontaneously self-annealing, alleviating the requirement for host-cell DNA synthesis. Additionally, different AAV vectors may also be selected based on different serotypes. AAV serotypes 2 and 9 (AAV2 and AAV9, respectively) are two examples of AAV vectors that may be employed in examples according to this disclosure.

In another example according to this disclosure, a herpes simplex viral (HSV) vector is employed to transduce a genetic agent encoding for an opsin into cells within patient 6 not only at the delivery site, but also to cells in a plurality of regions within the patient proximal and remote to the delivery site. In other examples, one of a number of different types of lentivirus vectors may be employed to transduce a genetic agent encoding for opsin(s) into target cells within patient 6.

A variety of techniques may be employed to deliver the viral vector with retrograde and/or anterograde transport properties and including the genetic agent encoding for an opsin to target site 24 within patient 6 (200). In one example, the viral vector is metered by an external infusion pump and delivered to patient 6 by percutaneous injection. For example, an AAV vector may be injected intracranially using stereotactic coordinates, micropipette 22 and an automated external infusion pump (not shown in FIG. 1) connected to the micropipette for precise delivery of AAV to target site 24 within brain 16 of patient 6 with minimal damage to tissue surrounding the delivery site. In another example, a viral vector may be delivered to target site 24 (or another site) within patient 6 intravenously. For example, an AAV vector may be delivered to sites within brain 16 of patient 6 via injection into the carotid artery (CA). In the case of intravenous delivery of the vector, a particular variety of virus may be selected, e.g. AAV9, which differs from previous AAV vectors in that it has a unique outer protein coat, or capsid, which may enable the AAV9 vector, unlike others, to enter the brain from the vasculature, crossing the blood-brain barrier and delivering a genetic agent encoding for an opsin widely throughout the brain.

Whatever viral vector is employed and whatever technique is used to deliver the vector to a target site within a patient (200) in the example method of FIG. 7, a variety of genetic agents encoding for light-sensitive protein(s), referred to herein as “opsins,” may be delivered by the vector. Delivering the genetic agents encoding for one of a number of appropriate opsins permits modulation of the transduced cells via optical stimulation. For example, the activation or inhibition of neurons transduced with DNA encoding for an opsin and thereby producing the opsin protein may be controlled by directing light of varying wavelengths and intensities at the cells. The devices and techniques described in this disclosure may be used in conjunction with any of a variety of opsins or other materials effective in supporting excitation, inhibition or other desired effects on the target tissue, e.g. of target neurons. In one example, a first opsin or set of opsins, also referred to as a light-activated cation channel proteins (or “LACC”), comprises the protein, or portions of the protein Channelrhodopsin-2 (ChR2). The opsins employed in examples according to this disclosure may also cause the modulation of the flow of anions such as chloride across a membrane when activated by light. In one example, a second opsin or set of opsins, also referred to as an anion pump, may comprise the protein, or portions of the protein, halorhodopsin (NpHR). A number of other example opsins may be employed including, e.g., archaerhodopsin-3 from Halorubrum sodomense (Arch), archaerhodopsin from Halorubrum strain TP009 (ArchT), and a blue-green light-drivable proton pump from the fungus Leptosphaeria maculans (Mac).

In some examples according to this disclosure, the viral vector delivered to target site 24 within patient 6 (200) may include a genetic agent encoding for one opsin, while, in other examples, the vector may include a genetic agent encoding for more than one opsin. In one example according to this disclosure, a first opsin may be employed as an activating or exciting opsin that, when exposed to a specific wavelength of light or range of wavelengths from light source 63 via optical fibers 11 via optical stimulation generator 60 of stimulator 4, causes the target neuron membrane to become permeable to cations into the neuron, which depolarizes the neuron, also referred to as activating the neuron, and causes a neural spike. A second opsin may be employed in addition to or in lieu of the first activating opsin as an inhibiting opsin that, when exposed to a different wavelength of light or range of wavelengths from light source 63 (or another light source included in or separate from stimulator 4) via optical fibers 11, acts to hyperpolarize the neuron, also referred to as inhibiting or deactivating the neuron, to counteract the cation permeability of the target neuron.

In one example of the method of FIG. 7, an AAV vector is delivered to the hippocampus of one hemisphere of brain 16 of patient 6. The AAV vector is injected intracranially using micropipette 22 and an automated external infusion pump (not shown in FIG. 1) connected to the micropipette for precise delivery of AAV to the hippocampus with minimal damage to tissue surrounding the delivery site. The AAV delivered via intracranial injection includes a nucleic acid sequence encoding for at least one of Channelrhodopsin-2 (ChR2), halorhodopsin (NpHR), archaerhodopsin-3 from Halorubrum sodomense (Arch), archaerhodopsin from Halorubrum strain TP009 (ArchT), and a blue-green light-drivable proton pump from the fungus Leptosphaeria maculans (Mac). Additionally, the AAV vector includes retrograde and/or anterograde transport properties such that the nucleic acid encoding for the opsin is transduced not only into cells at the delivery site in the hippocampus, but also to cells with axons or dendrites or other cell parts at the delivery site in the hippocampus but whose cell bodies are in a plurality of regions within the patient proximal and remote to the delivery site. For example, the AAV vector may transduce the nucleic acid encoding for the opsin into cells at various ipsilateral regions in the cerebral cortex and/or contralateral regions in the hippocampus in the other hemisphere of brain 16 of patient 6, as described in the illustration of FIGS. 2A and 2B. In this manner, examples according to this disclosure may deliver genetic agents encoding for opsins into a number of regions of a patient's brain through minimally invasive surgical techniques that target a single delivery site. Such efficient and less invasive techniques may enable realization of the clinical potential of targeted activation of light-sensitive proteins in transduced cells in a wide range of regions of a patient's body via optical stimulation to modulate cellular behavior and, thereby, treat various neurological conditions, including, e.g., depression, dementia, obsessive-compulsive disorder and movement disorders, such as Parkinson's disease, spasticity, epilepsy, and dystonia.

In addition to delivering a viral vector comprising a genetic agent encoding for one or more light-sensitive proteins to a target site within a patient (200), the method of FIG. 7 includes sensing a bioelectrical signal related to a neurological condition of the patient (202). In one example, the sense electrodes associated with electrical leads 12 connected to stimulator 4 may detect various types of bioelectric signals, including, e.g., local field potentials (LFP) of brain tissue, energy spectra in different bands, such as alpha, beta, or gamma bands of brain activity, and electrical signals associated with electrocorticography (ECoG) or electroencephalography (EEG). In one example, sense electrodes on leads 12 may be employed by stimulator 4 to predict the onset or detect the occurrence of a seizure related to or caused by a neurological condition of patient 6. For example, stimulator 4 may be configured to employ leads 12 to sense one or more bioelectrical signals, e.g. LFP, ECoG, and/or EEG in order to predict the onset or detect the occurrence of an epileptic seizure.

In one example, stimulator 4 may be configured to execute one or more algorithms to predict the onset or detect the occurrence of an epileptic seizure based on one or more bioelectric signals detected by sense electrodes on leads 12. In one example, seizure prediction and/or detection algorithms are stored on memory 52 and executed by processor 50, which may also control sense electrodes on leads 12. One type of seizure detection algorithm that may be executed by processor 50 of stimulator 4 indicates a seizure upon sensing of a bioelectrical brain signal that exhibits a certain characteristic, which may be a time domain characteristic (e.g., an amplitude) or a frequency domain characteristic (e.g., an energy level in one or more frequency bands). For example, processor 50 may execute a seizure detection algorithm stored on memory 52 that indicates a seizure is detected when the amplitude of bioelectrical brain signal sensed by electrodes on leads 12 meets a certain condition relative to a threshold (e.g., is greater than, equal to or less than the threshold), which threshold may be stored, e.g. on memory 52. In another example, processor 50 may execute a seizure detection algorithm stored on memory 52 that detects a seizure onset if a sensed bioelectrical brain signal substantially correlates to a signal template (e.g., in terms of frequency, amplitude and/or spectral energy characteristics), which template may also be stored on memory 52. Additional information regarding detecting the occurrence or predicting the onset of seizures is described in U.S. application Ser. No. 11/799,051, filed Apr. 30, 2007 entitled “SEIZURE PREDICTION,” and U.S. application Ser. No. 12/432,268, filed Apr. 29, 2009 and entitled “SEIZURE DETECTION ALGORITHM ADJUSTMENT,” the entire contents of both of which are incorporated herein by this reference.

The example method of FIG. 7 also includes delivering optical stimulation to one or more cells transduced with the genetic agent by the viral vector to treat the neurological condition of the patient (204). In one example, stimulator 4 delivers optical stimulation to target neurons within brain 16 of patient 6 that have been transduced with a genetic agent encoding for one or more opsins by an AAV vector delivered to the hippocampus in one hemisphere of the brain. In such an example, although the vector has been delivered to the hippocampus in one hemisphere of brain 16, stimulator 4 may nevertheless deliver optical stimulation to target neurons in a number of other locations within the brain due the retrograde and/or anterograde transport properties of the AAV vector that enable it to transduce the genetic agent to not only the hippocampus delivery site, but also to a number of locations proximal and remote to the delivery site.

The optical stimulation delivered by stimulator 4 may be configured to activate the opsins in order to modulate the behavior of the target neurons, e.g. to modulate the action potential to activate and/or inactivate the neuron cells. In one example, as described above, a first opsin is activated by a first wavelength of light delivered by stimulator 4 via optical stimulation generator 60, light source 63 and fibers 11 so that the target neurons become permeable to cations to initiate neuronal spikes and fire the target neuron and a second opsin is activated by a second wavelength of light to deactivate or inhibit the target neurons. In this example, stimulator 4 may provide for a useful chronic device enabling continuous therapy by providing light at both the first wavelength and the second wavelength on an alternating or selective basis in order to provide for both selective activation and inhibition of the target neurons.

For example, if ChR2 is used as the activation opsin and NpHR is used as the inhibition opsin, then optical stimulation generator 60, and particularly light source 63, may be controlled by processor 50 to provide light of a first wavelength of between about 420 nm and about 475 nm, such as about 450 nm, to activate the ChR2 and activate the target neurons and a second wavelength of between about 510 nm and about 580 nm, such as about 535 nm, to activate the NpHR and inhibit the target neurons. In another example, optical stimulation generator 60 may be configured to stimulate target neurons within brain 16 of patient 6 to open ChR2 channels via brief pulses of light with a wavelength of about 450 nm with an intensity of between about 8 mW/mm² and about 12 mW/mm² and a duty cycle of about 1% and a pulse frequency of between about 100 Hz and about 120 Hz, such as a 100 μs pulse every 10 ms. In one example, an NpHR opsin is activated, thus deactivating the target neuron, when exposed to yellow light from light source 63 via fibers 11 by optical stimulation generator 60 having a wavelength of between about 550 nm and about 610 nm, such as between about 570 nm and about 590 nm, and in one example about 580 nm.

In some examples, because light transmitted from light source 63 to tissue within brain 16 of patient 6 may scatter and because the required voxel size for therapy may not be well established, optical stimulation generator 60 may be designed to be scaleable with the ability to incorporate multiple stimulation circuits and outputs to increase the volume of activation by using multiple pathways. In another example, one or more lenses may be used to focus or dissipate the light, as desired to provide more intense or scattered optical stimulation in the target tissue. In one example, a lens may be created at distal ends optical fibers 11 by modifying or distorting the distal end of each fiber to form a lens that may focus or dissipate light once it reaches the distal end.

Optical stimulation generator 60 may provide for the two wavelengths of stimulation light by having two light sources, one for each wavelength, such as a LED dedicated to each wavelength of light that feed into optical fibers 11 to deliver each wavelength of light to the target neurons depending on which LED is activated by optical stimulation generator 60 as controlled by processor 50. Alternatively, a single light source 63 that is capable of emitting both wavelengths may be used, such as a tunable LED or other tunable light source, wherein a particular wavelength is selected based on a treatment program run by processor 50 that causes optical stimulation generator 60 to control light source 63 to emit the selected wavelength, such as by tuning the tunable LED or other tunable light source to the selected wavelength.

Optical stimulation generator 60 may also control, under the direction of processor 50, several other parameters with respect to the optical stimulation of the target tissue, such as the intensity of light emitted to stimulate the target tissue, the number of pulses of light to be emitted, the pulse width, the frequency of pulses, and the pattern of pulses, including burst patterns wherein optical stimulation generator 60 may control burst width, burst frequency, the number of pulses per burst, and the number of bursts. In one example, optical stimulation generator 60 is capable of delivering light from light source 63 via one or more of optical fibers 11 with an intensity of between about 1 mW/mm² and about 5 mW/mm². When light pulses are used as part of a stimulation program, optical stimulation generator 60 may, for example, produce pulses with a pulse width of between about 100 μs and about 15 ms, such as pulses with a pulse width of about 10 ms, and with a pulse frequency of between about 0.1 Hz and about 1 kHz, such as a frequency of about 0.2 Hz. In one example, optical stimulation generator 60 may drive optical stimulation with programmable modulation patterns that mimic existing patterns used in electrical stimulation for deep brain stimulation (DBS), and also allow for novel patterns that leverage the capabilities of the inhibitory optical transducer in the cell membranes.

In examples including the treatment of epilepsy, the determination of the foci of the epileptic seizure may be difficult. As such, techniques may be employed to enable optical stimulation to a number of different locations within brain 16 of patient 6, in order to iteratively narrow the focus of stimulation, e.g. through trial and error, to a particular location from which or close to which the seizure emanates. In one example, stimulator 4 may be connected to optrode array 82 of the example of FIG. 4, on which a number of optrodes 84 are located. Optical stimulation generator 60 may be controlled by processor 50 to direct one or more light sources from light source 63 through one or more fibers bundled in lead body 10 to selectively activate one or more of optrodes 84. In this manner, optrodes 84 of optrode array 82 may be activated in a controlled manner in a number of different locations within brain 16 of patient 6, e.g. within the cerebral cortex to iteratively narrow the focus of optical stimulation to one or locations from which to close to which an epileptic seizure emanates.

In some examples according to this disclosure, stimulator 4, or another such implantable or external medical device, may be configured to employ one or more sensors to deliver closed loop, adaptive optical stimulation to patient 6. For example, in addition to employing electrodes on leads 12 to sense bioelectrical signals in order to predict the onset or detect the occurrence of a seizure, the sense electrodes may also be controlled, e.g. by processor 50 to adapt optical stimulation delivered to patient 6 based on changes in the bioelectric signals. For example, changes in a bioelectric signal or a number of signals, e.g. LFP, ECoG, and/or EEG, received by processor 50 of stimulator 4 may indicate the movement of the foci of an epileptic seizure from one region of brain 16 of patient 6 to another. In such an example, processor 50 may adaptively alter the selected optical fibers 11 or optrodes 84 from which to deliver optical stimulation to neurons within brain 16 based on the sensed bioelectric signal or signals.

Although the target therapy delivery site described with reference to the foregoing examples is within the brain of a patient, other applications of therapy systems in accordance with this disclosure include alternative delivery sites. In some examples, the target delivery site may be proximate to different types of tissues including, e.g., nerves, e.g. sacral, pudendal or perineal nerves, organs, muscles or muscle groups. In one example, a catheter may be positioned to deliver a therapeutic fluid to a site proximate the spinal cord or within the heart or blood vessels. A catheter may also be positioned to deliver insulin to a patient with diabetes. In other examples, the system may deliver a therapeutic fluid to various sites within a patient to facilitate other therapies and to manage other conditions including peripheral neuropathy or post-operative pain mitigation, ilioinguinal nerve therapy, intercostal nerve therapy, gastric drug induced stimulation for the treatment of gastric motility disorders and/or obesity, and muscle stimulation, or for mitigation of peripheral and localized pain e.g., leg pain or back pain.

The techniques described in this disclosure for delivering optical stimulation may be implemented, at least in part, in hardware, software, firmware or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

### EXAMPLES

Several animal studies were undertaken to evaluate the ability of adeno-associated viral (AAV) as a vector to transduce a genetic agent (such as an agent encoding for light-sensitive protein(s)) to cells not only at a delivery site within a subject at which the vector is initially delivered, but also to cells in a plurality of regions within the subject proximal and remote to the delivery site. Stated objectives of the studies were: 1) to determine whether AAV9 can be used to deliver DNA to the brain of adult animals from a cranial artery route of delivery, when either used alone or combination with an agent that temporarily opens the blood-brain barrier, and if found to be so in small animals, 2) to determine whether this capability can be scaled up to a larger animal. All viral vectors used in the following studies were purchased from ReGenX, Inc., Washington, D.C.

Study 1: Preliminary Study of scAAV9 Delivery to the Brain Via Carotid Artery Infusion, in Mice

This study sought to confirm and extend the findings of Duque et al. (2009), who reported that self-complementary AAV9 (a form of the vector in which the DNA payload carried inside the capsid folds back onto itself) can not only deliver DNA to the central nervous system of neonatal mice (in which the blood-brain barrier has not yet fully formed), but also can deliver DNA to the spinal cord and some brain cells of adult mice and adult cats, upon intravenous delivery. The self-complementary form of AAV is thought to lead to better transduction of cells by the virus, because only one copy of the viral DNA provides the cell with a “ready to use” double-stranded DNA, rather than requiring the cell to take up two copies (one strand from one viral particle, and the complementary strand from another viral particle) or replicate the single copy, to yield a functional DNA.

A self-complementary AAV9 was employed expressing a reporter gene, enhanced Green Fluorescent Protein (eGFP). The study focused on delivery of this reporter gene to the brain, rather than the spinal cord. Due to the high cost of the scAAV9-eGFP, the dose of the vector was limited to 3×10e11 vector genomes (vg) per mouse, the lowest of the dosages reported by Duque et al. (2009), whose dosages ranged from 3×10e11 to 2×10e12 vg per mouse, injected into the tail vein. In this study, the scAAV9 was injected into the carotid artery, allowing for first-pass delivery of the vector into the brain vasculature. In addition, in some mice, prior to the infusion of the scAAV9, a 1.8 Molar (M) solution of arabinose was infused to open the blood-brain barrier, to determine whether this would further enhance the ability of the scAAV9 to enter the brain.

Seventeen mice were used for the study. All mice were pre-catheterized by surgeons at Charles River Laboratories, to have a catheter positioned in the left internal carotid artery, with the catheter tip pointed antegrade with the blood flow (i.e., towards the brain). In one group of mice, 300 micro liters (μl) of 1.8 M arabinose (in water, at room temperature) was delivered through the catheter at 600 μl/minute. Five minutes later, 280 μl of scAAV9 at 1.08×10e9 vg/μl was delivered at 600 μl/minute, for a total of 3.02×10e11 vg/mouse. In a second group of mice, only the latter infusion comprising the 3.02×10e11 vg of scAAV9 was delivered. Any mouse whose catheter was found to be non-patent upon initial catheter access (based on lack of visible blood and resistance to a gentle attempt at a saline flush of the catheter) was deferred to the control group of animals, which received a stereotactic infusion of 3×10e8 vg of scAAV9 in 2.8 μl of volume, into the right hippocampus. The stereotactic coordinates for the hippocampal infusion were, from bregma: AP −2.7 mm, ML −3.0 mm, and DV −1.75 mm below dura.

To counterbalance the possibility that the eGFP reporter gene expression might be limited due to its being driven by the CMV promoter in the scAAV9 vector, mice in each experimental condition were terminated at two different time points, an early time point (10 to 11 days), and later time point (14 to 15 days) post-virus delivery. (The results showed that precaution was unnecessary, and thus it was dropped in all further studies).

One mouse in the arabinose+scAAV9 group, scheduled for termination after 15 days, instead died two days after the vector delivery, and was eliminated from the study. A second mouse in this group, scheduled for termination after 10 days, was found dead in its cage after six days; however its brain was recovered, and included in the study (although the tissue did not section well).

The resulting experimental groups and number of mice per group in this study are summarized below in Table 1.

TABLE 1

| | Time point | |
|---|---|---|
| | of termination | |
| N mice per group (16 total survivors) | post-viral delivery | |
| Experimental condition | 10-11 days | 14-15 days |
| scAAV9 delivered via carotid artery | 3 | 3 |
| arabinose followed by scAAV9, | 3 | 2 |
| via carotid artery | | |
| scAAV9 via hippocampal injection | 3 | 2 |

On the appropriate termination date, each mouse was transcardially perfused with saline followed by 4% paraformaldehyde. The brains were removed and sent to Neuroscience Associates (Nashville, Tenn.) for sectioning coronally in a multi-brain block, allowing the tissue from all animals in the study to be stained identically. The tissue was immunostained for eGFP protein, to reveal cells transduced by the virus. The eGFP immunostain was visualized using diaminobenzidine (DAB, brown color reaction) with alternating slides visualized using nickel-DAB (resulting in an intense black stain).

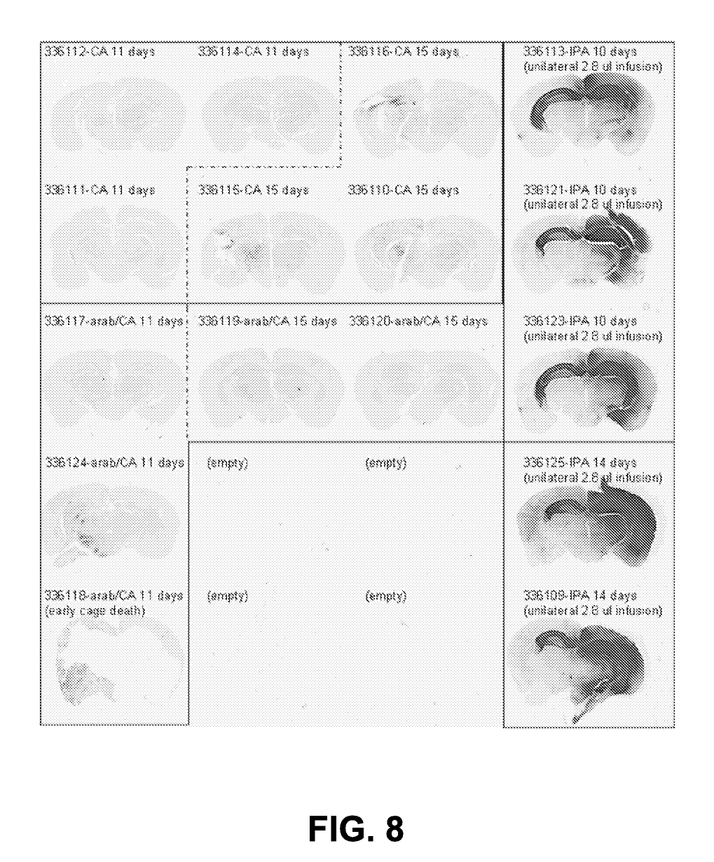

A representative section of the mouse brains through the region of the hippocampus, immunostained for eGFP, is shown in FIG. 8, with the experimental conditions for each of the mice annotated on the figure.

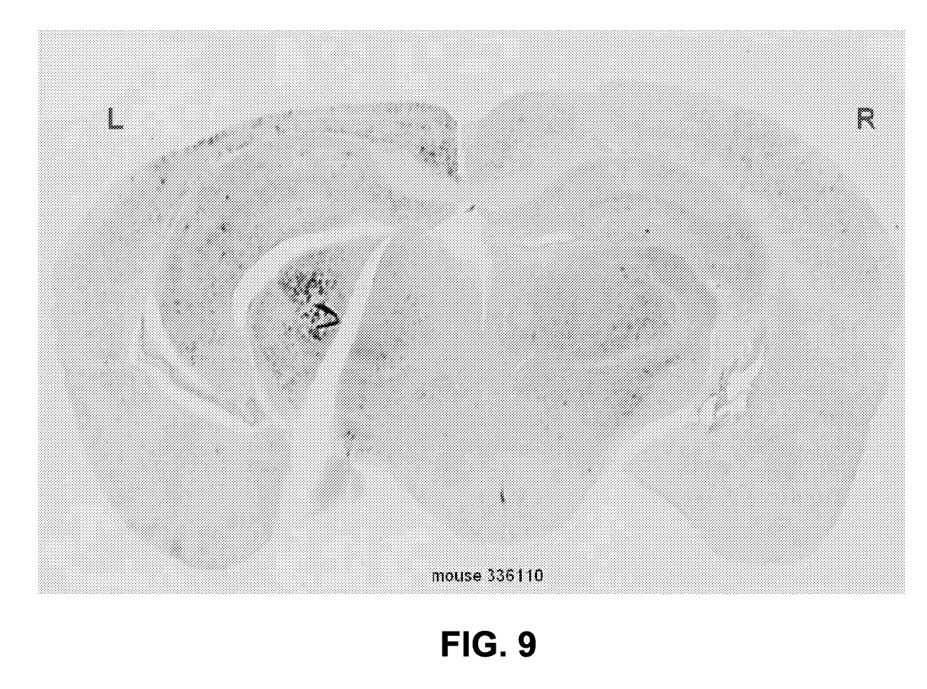

Qualitatively, there did not appear to be an appreciable difference between mice in a given experimental condition with regards to the early versus later time point of termination post-viral delivery. Based on these results, the concern that the transgene expression from the CMV promoter might be “shut down” after two week's time in the mouse brain was unfounded or, at the least, of reduced concern. Microscopic examination of the brain sections readily revealed eGFP-positive cells throughout the brains in all mice receiving the scAAV9 via carotid artery infusion. Consistent with the delivery of the vector into the left carotid artery, there was a tendency for a greater density of eGFP-positive cells to be found in the left versus the right hemisphere; however, the transduction of cells was not limited to the left hemisphere. FIG. 9 provides a somewhat higher magnification view of a representative coronal section from a mouse receiving scAAV9 through the carotid artery, without pre-administration of arabinose.

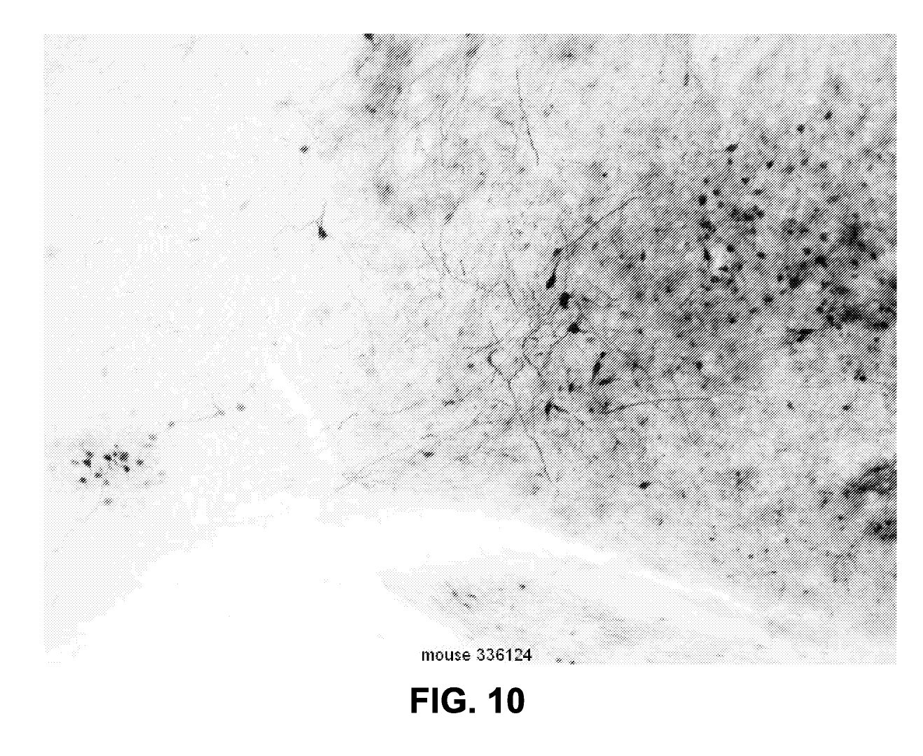

An important question about the transduced cells apparent in these brains is whether they include brain cells, and particularly neurons, indicating true passage of the viral vector across the blood-brain barrier, and are not limited merely to endothelial cells lining the brain's blood vessels. FIG. 10 illustrates the morphology of at least a proportion of the transduced cells in the brain of a mouse receiving scAAV9 via the carotid artery after pre-treatment with arabinose. The morphology of the stained cells (here, visualized using nickel-DAB), along with their anatomical clustering, is indicative of neuronal cells, as both neuropil (axons and/or dentrites) and localization of the cell bodies in nuclei is evident.

Further examination of the tissue sections led to the qualitative impression that in the animals receiving pre-treatment with arabinose, there was a tendency for more of the transduced cells to be neurons as well as astrocytes, whereas in the mice receiving the scAAV9 via the carotid artery without pre-treatment with arabinose, astrocytic cells more prevalent among transduced cells than neurons. Nevertheless, it is apparent that pre-treatment with arabinose was not a prerequisite for the scAAV9 administration via the carotid artery to result in transduction of cells in the brain, and not just the cerebral arteries.

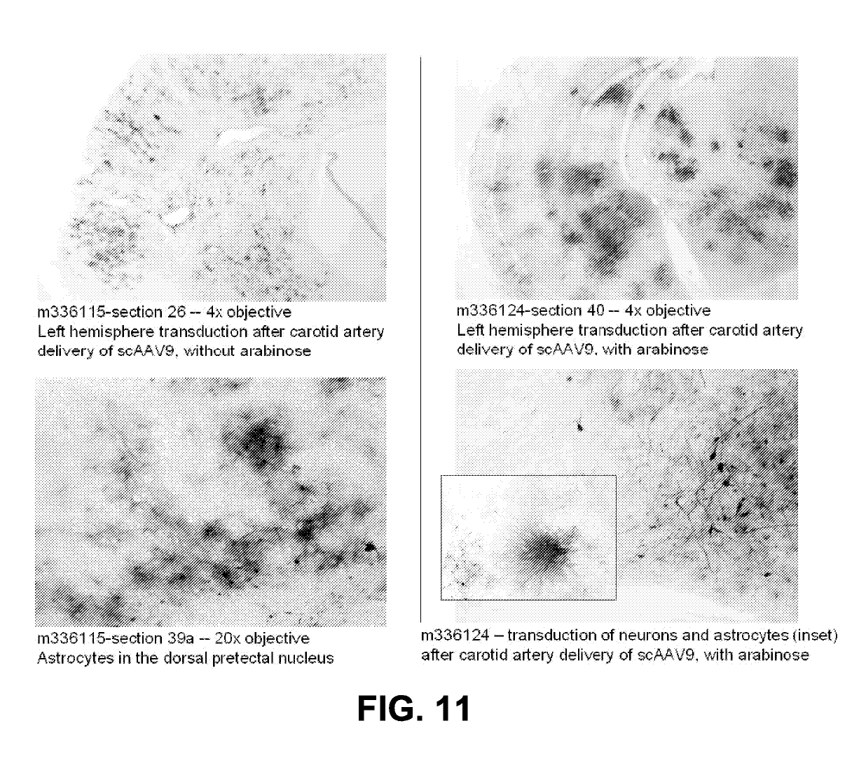

In all cases, the transduction of cells in the brain in mice receiving scAAV9 through the carotid artery was bilateral, and also extensive along the rostral-caudal axis, as illustrated in FIG. 11, which includes coronal sections of mice receiving scAAV9-eGFP by left carotid artery infusion, stained for eGFP and visualized with nickle-DAB. However, it was also regionally uneven, with some regions of the brain evidencing more cellular transduction than other regions, in an apparently random fashion. Comparison of these results to those in the second study (detailed below in Study 2), suggests that the “unevenness” is partially a function of the dosage of the viral vector used, because a higher dosage resulted in somewhat more “even” transduction.

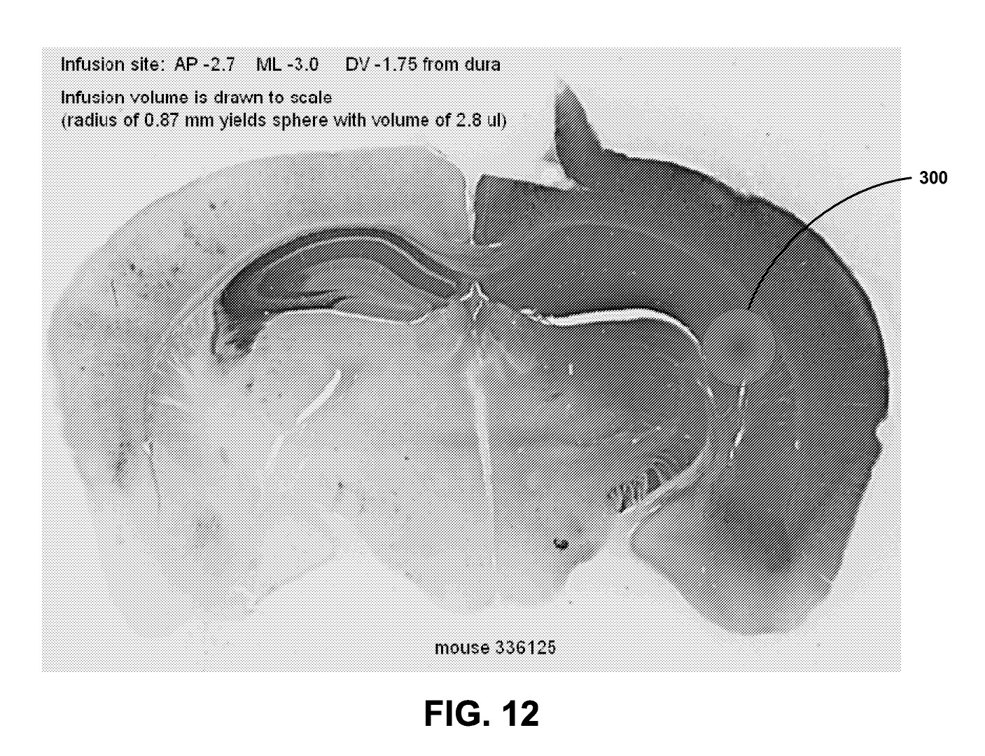

Unexpectedly, the results observed in all five of the mice in the control group, receiving a stereotactic infusion of 1/1000 of the viral particles (3×10e8 vg/mouse) in a small volume (2.8 μl), showed transduction of cells extending far beyond the region of the infusion in the right hippocampus. FIG. 12 shows the results obtained in one mouse, with the equivalent volume 300 of the infusion (if the infusion resulted in a spherical distribution) superimposed on the photograph, and drawn to scale.

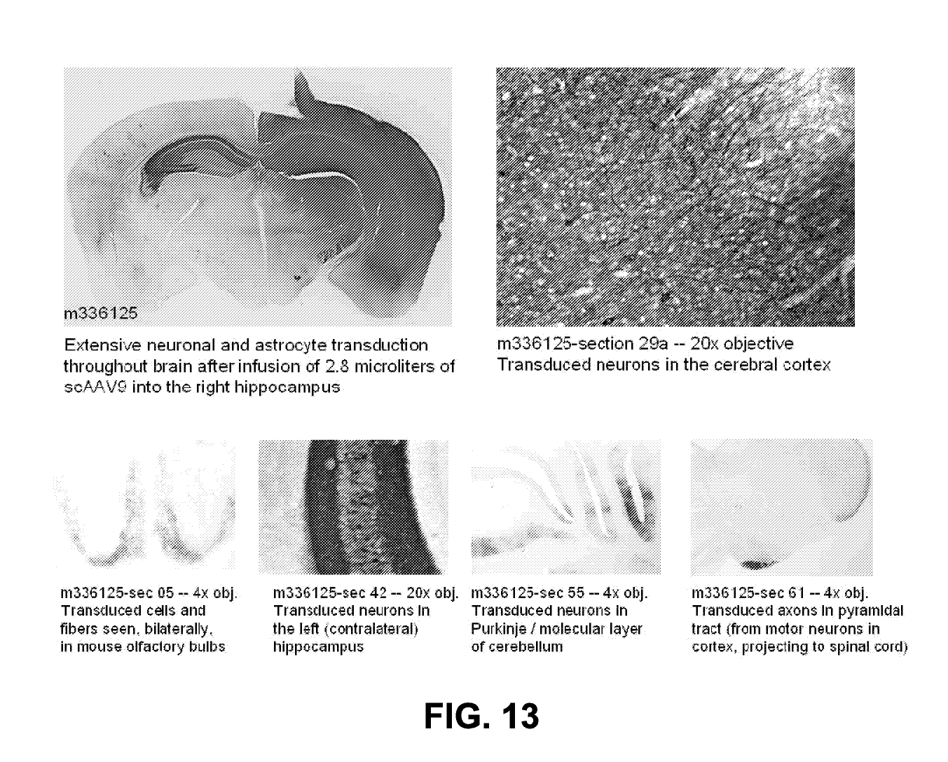

As shown in FIG. 13, the transduced cells extended throughout the rostral-caudal axis of the mouse, with transduced cells seen in every coronal section throughout the brain. Transduced tissue included both fibers and cells in the olfactory lobes, bilaterally, and also Purkinje neurons in the cerebellum, also bilaterally. In addition, there was transduced tissue throughout the ipsilateral cerebral cortex, far greater than could be expected from “leakage” of the viral infusion up the needle track. Transduced tissue in the cortex included neuronal cells bodies, suggesting retrograde transport of the scAAV9 from the infusion region in the hippocampus up the axons of cortical neurons projecting to the hippocampus. Consistent with this interpretation is the extensive transduction of neurons in the contralateral hippocampus, also indicating retrograde transport of the viral vector via axons projecting from the contralateral hippocampus to the ipsilateral side.

The unexpectedly extensive transduction resulting from the intraparenchymal infusion of the scAAV9 was observed in five out of five animals in the control group (see FIG. 8), suggesting that phenomenon was consistent. However, from this study alone, it could not be determined whether the unexpected result was due to unique properties of AAV serotype 9, the greater transduction efficiency resulting from a self-complementary rather than “standard” (single-stranded) AAV vector, or the sensitivity of the eGFP immunostaining utilized. Therefore, along with replicating the blood-brain barrier passage of the scAAV9 with a higher viral dose, Study 2 described below was designed to address this issue.

Study 2: Confirmatory Study of scAAV9 Delivery to the Brain Via Carotid Artery Infusion, in Mice

Three viral vectors, all encoding eGFP expressed from the CMV promoter, were used in this study. The first was the same scAAV9-eGFP used in study 1. The second was a self-complementary AAV of serotype 2 (scAAV2-eGFP), and the third was a standard, single-stranded AAV2-eGFP.

The viral vectors were administered by either the carotid artery or intraparenchymal route of injection. Because there is ample evidence from prior literature that standard AAV2 does not enter the brain from the vasculature, this vector was not administered to animals via the carotid artery. The other two vectors were each administered to six pre-catheterized mice via the carotid artery, in 300 μl volume at 600 μl/minute, at a viral titer of 2×10e9 vg/μL. Thus, the dosage of virus administered via the carotid artery was 6×10e11 vg/mouse, twice the dosage used in Study 1.

All three vector types were administered to mice via stereotactic injection into the right hippocampus. However, due to differences in viral titer of the available lots of vector, the dosage and volume injected varied from 3×10e9 vg in 2.8 μl of volume (replicating Study 1) to 1.7×10e9 vg in 6 μl of volume (the maximum volume advisable in a stereotactic injection into mouse brain per injection site). Intraparenchymal injections were all performed at a rate of 0.5 μl per minute.

The number of mice per group, and the viral type, dosage, and volume received are summarized below in Table 2.

TABLE 2

| | | Carotid Artery Infusions | | | Intraparenchymal injections | | |
|---|---|---|---|---|---|---|---|
| | | (left carotid artery) | | | (right hippocampus) | | |
| | | | | volume | | | volume |
| Viral type | Lot | N mice | vg admin | (μl) | N mice | vg admin | (μl) |
| scAAV9-eGFP | V1467 | 6 | 6 × 10¹¹ | 300 | 2 | 1.7 × 10⁹ | 6.0* |
| | V1084 | | | | 2 | 3.0 × 10⁹ | 2.8** |
| | V1084 | | | | 2 | 1.9 × 10⁹ | 6.0*** |
| scAAV2-eGFP | V1465 | 6 | 6 × 10¹¹ | 300 | 4 | 1.9 × 10⁹ | 6.0 |
| AAV2-eGFP | V1455 | | | | 3 | 1.9 × 10⁹ | 6.0 |

*The titer of this lot of scAAV9-eGFP limited the dosage attainable in the mice receiving the intraparenchymal injections.
**These two mice received a dose and volume replicating that of study 1.
***These two mice received a dose and volume for direct comparison to the other scAAV2-eGFP and AAV-eGFP groups.

All mice were terminated on the same day, which was 15 days post-viral infusion for the mice in the carotid artery groups, and 14 days post-viral injection for the mice in the intraparenchymal groups. Each mouse was transcardially perfused with saline followed by 4% paraformaldehyde. The brains were removed and sent to Neuroscience Associates (Nashville, Tenn.) for sectioning and staining as in the first study described above in Study 1.

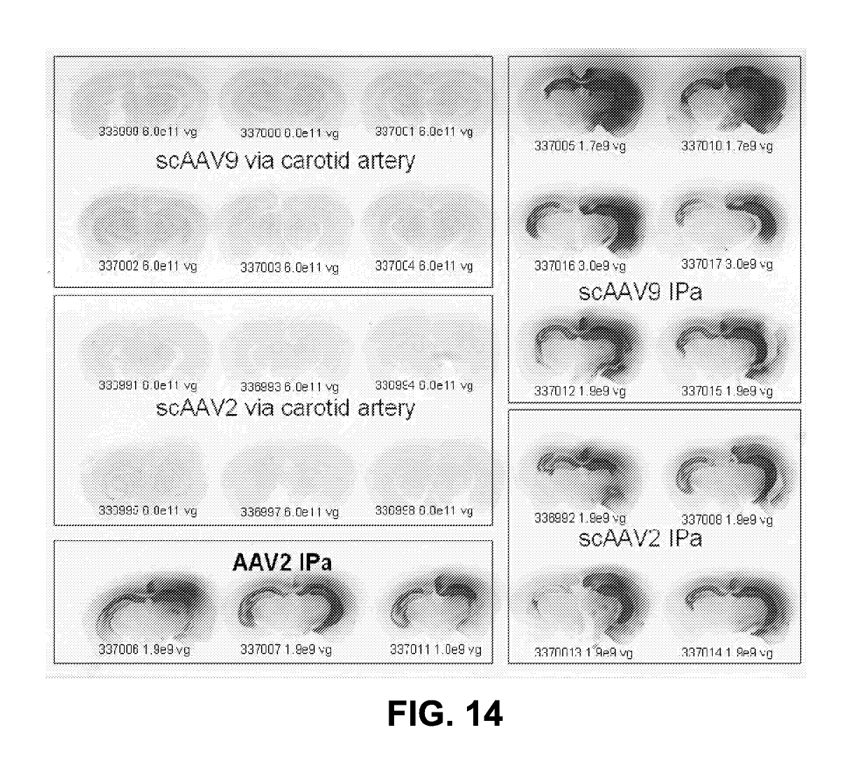

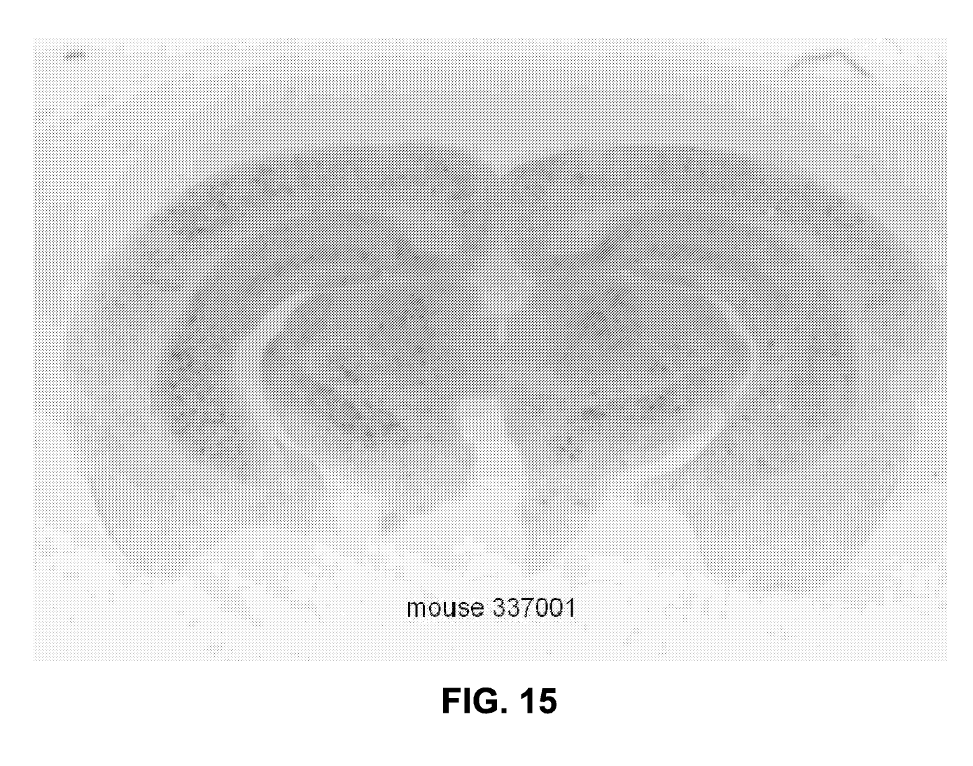

An overview of the results of this study is provided by FIG. 14, which is a multi-brain block showing coronal sections of all mice in the second study, immunostained for eGFP protein expression. FIG. 14 is annotated with mouse number and viral dose. In all six out of the six mice receiving scAAV9-eGFP via carotid artery infusion, eGFP transduced cells could be seen in the brain. FIG. 15 provides a higher magnification view of a coronal section of a mouse receiving scAAV9-eGFP via carotid artery infusion and illustrates widespread distribution of eGFP-transduced cells in a mouse receiving scAAV9-eGFP via carotid artery infusion (visualized by nickle-DAB).

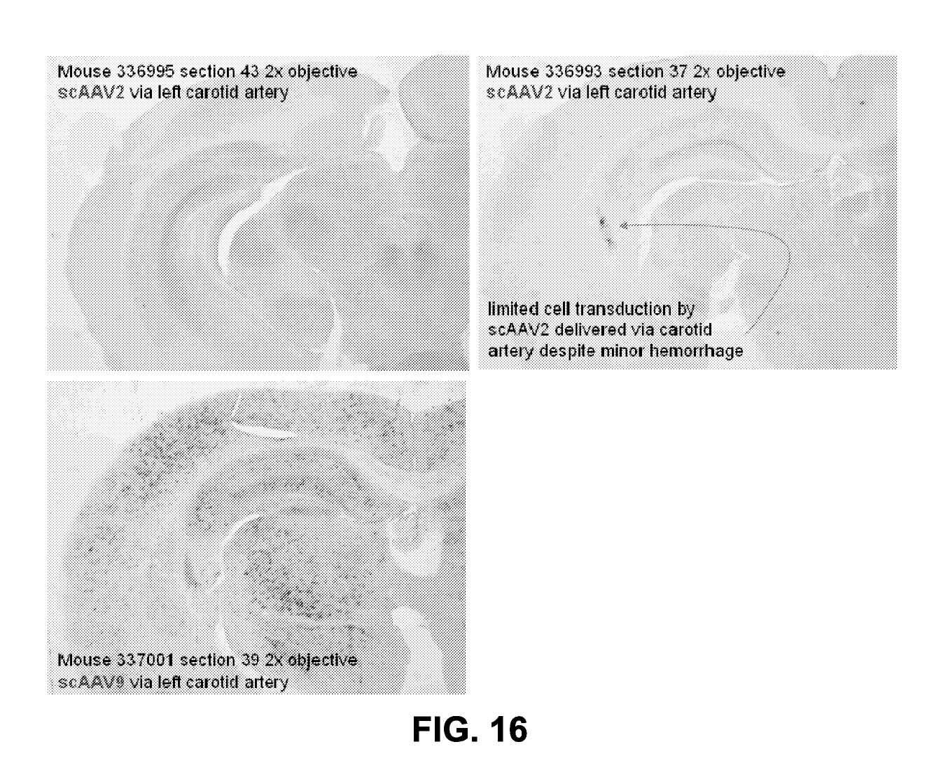

Conversely, minimal staining for eGFP was seen in each of the six mice receiving the same dosage of scAAV2 viral particles via carotid artery infusion. This was the case even in the event of apparent compromise of blood-brain barrier integrity due to a minor cerebral hemorrhage, as shown in FIG. 16, which compares eGFP staining results in mice receiving scAAV9 versus scAAV2. As illustrated in FIG. 16, scAAV2 does not cross the blood-brain barrier to widely transduce cells (see top left panel), even in the case of a small hemorrhage in the brain (see top right panel). In contrast, as illustrated in the bottom left panel in FIG. 16, there was widespread transduction of cells throughout the brain in all six of six mice receiving scAAV9.

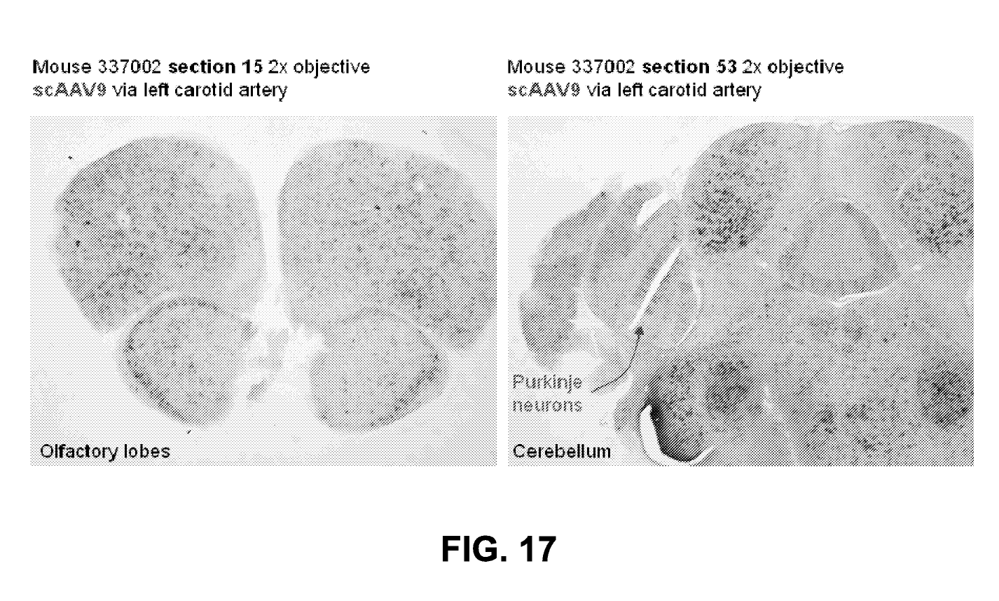

In the mice receiving the scAAV9-eGFP by carotid artery infusion, the transduction of cells with eGFP occurred bilaterally throughout the brain, including the entire rostral-caudal extent, as shown in FIG. 17, which illustrates widespread distribution of eGFP-transduced cells in mice receiving scAAV9-eGFP via carotid artery infusion (visualized by nickle-DAB). Transduction of cells by scAAV9 delivered via carotid artery infusion was found throughout the brain, bilaterally, in all six of six mice receiving scAAV9. In FIG. 17, the left panel shows extensive transduction in the olfactory lobes of a mouse receiving scAAV9. The right panel in FIG. 17 shows extensive transduction of cells, including Purkinjie neurons, in the cerebellum of the same mouse.

In addition, qualitatively, the distribution of transduced cells in the brains of the mice in this study receiving 6×10e11 vg of scAAV9-eGFP via carotid artery infusion appeared to be less “spotty” in the brain, than in the mice in the first study that received 3×10e11 viral genomes of scAAV9-eGFP via carotid artery infusion. This observation suggests that with increasing dosages of the scAAV9, the transduction of cells in the brain may become more uniform.

Inspection of the coronal sections of the mice receiving the viral vectors by intraparenchymal injection into the right hippocampus (see FIG. 14) immediately reveals that transport of the vector from the ipsilateral hippcampus to the contralateral hippocampus occurred as readily with AAV2 and scAAV2 as with scAAV9, suggesting that there is no difference between the two serotypes nor between self-complementary and standard AAV in retrograde transport of this type. Conversely, in two of the six mice receiving scAAV9-eGFP into the right hippocampus, there appears to be substantially greater distribution of the eGFP protein in the injected hemisphere, including the cortical regions, compared to the mice receiving self-complementary or standard AAV2.

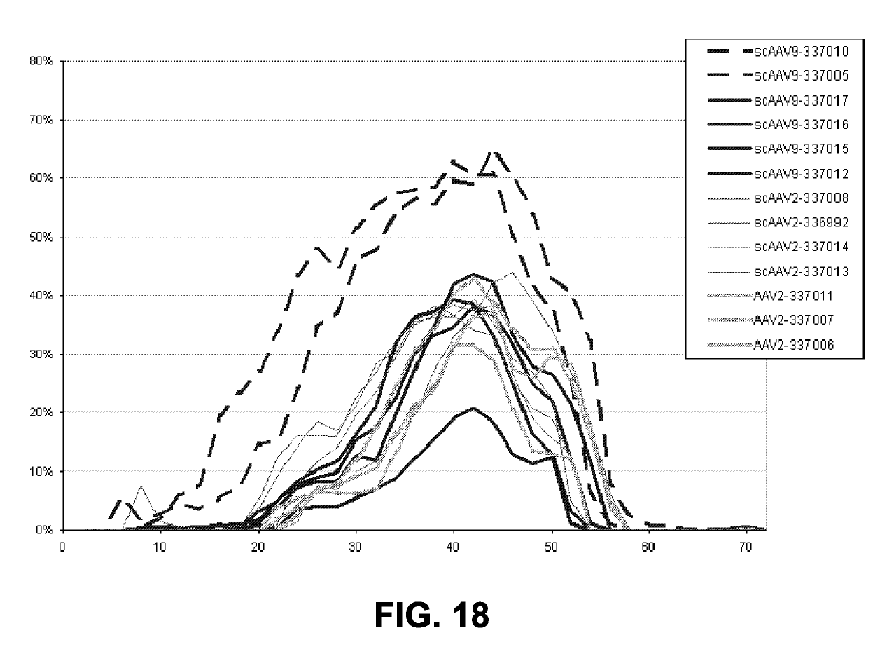

To determine whether this impression corresponds to a quantifiable difference, the amount of eGFP positive brain area in each mouse was quantified as follows. Images of the sections were digitized at a high resolution, and then converted to binary images based on a threshold brightness value to identify eGFP positive pixels. (The threshold was determined once manually, and then held constant across all sections and all slides.) Similarly, the images were converted to binary images based on a threshold brightness value that separates tissue from backgrounds; this threshold was held constant across all sections and slides. Finally, for each mouse and each coronal tissue section, the percent of cross-sectional area that is eGFP positive in each tissue section was computed as the count of eGFP positive pixels divided by the total number of pixels in the tissue section. The results are shown in FIG. 18, which graphs the percentage of eGFP positive cross-sectional area for each mouse, by Multi-brain block slide, going through the rostral-caudal extent of the brains.

It is apparent from the graph in FIG. 18 that two of the six mice receiving the scAAV9-eGFP did, in fact, have a substantially greater distribution of the viral transgene in the brain than all the remaining mice in the study (35.6% and 35.5% total volume [sum of cross-sectional areas] respectively, versus a maxium of 18.7% in all other mice). Remarkably, these two mice are not the two mice that received the highest dosage of the scAAV9-eGFP; to the contrary, they are the two mice that received the lowest dose, 1.7×10e9 vg. Note also that the viral lot received by these mice was different from the viral lot used in the first study described above in Study 1. Therefore, the increased distribution of the transgene in these mice cannot be accounted for as either a dosage effect or as an effect unique to a given lot of the virus. Together with the results of study 1 (compare FIG. 8 and FIG. 14), these results suggest that in some instances, an AAV of serotype 9 can distribute twice as widely from an intraparenchymal injection than would be expected for an AAV of serotype 2. However, this phenomenon is not consistent across all animals. Statistically, there was no difference found among the scAAV9, scAAV2, and AAV2 groups in terms of total volume of distribution of transduced brain tissue (measured as the sum of the cross-sectional areas in FIG. 18).

Further research would be required to determine whether the apparent tendency for scAAV9 to distribute more widely than the other two vectors is a true and reliable phenomenon. Based on the results obtained so far, to pursue this question in mice would require a study of 38 to 39 mice (2 groups of 19 mice each to compare serotypes, or 3 groups of 13 mice each to compare all three vectors) to have an 80% probability of detecting a true difference at the p=0.05 significance level.

Study 3: Generalization and Scale-Up Study of scAAV9 Delivery to the Brain Via Cerebral Artery and Via Direct Brain Infusion, in Sheep

The third study explored, in one sheep each, the delivery of scAAV9-eGFP to the sheep brain via a cerebral artery route and delivery by direct intraparenchymal injection.

In the sheep receiving scAAV9-eGFP via cerebral arterial infusion, viruses from three viral lots of scAAV9-eGFP (V1367, V1374, and V1467) were diluted to a common viral titer of 1.5×10e12 vg/mL in sterile saline. In order to maximize the probability of seeing an effect from arterial delivery, an effort was made to infuse the scAAV9-eGFP not into the carotid artery, but into a cerebral artery as far into the brain as possible. Under fluoroscopic guidance, a catheter was advanced from an entry point in the left common carotid artery in the neck of the sheep into a cranial artery. The use of Isovue to aid in the fluroscopic visualization of the catheter position was limited (a total of 43 mL of Isovue diluted 1 to 1.5 in warm saline was delivered), to minimize possible effects of the Isovue on the blood-brain barrier. Once the catheter was positioned, a polyurethane tube (Medtronic Model 10640 tubing) was inserted inside the positioned catheter, and used to deliver the viral vector. (This was to avoid potential unknown interactions between the virus and the catheter material used to access the cranial artery. Model 10640 has been used in prior sheep studies for delivery of AAV without detriment to the viral vector). A total of 2.5 mL of the viral vector at 1.5×10e12 vg/mL was delivered through the Model 10640 polyurethane, at a rate of 0.5 mL per minute, for a total delivered viral load of 3.75×10e12 vg. (No precedent for the appropriate viral dose for cranial artery delivery in a sheep existed.)

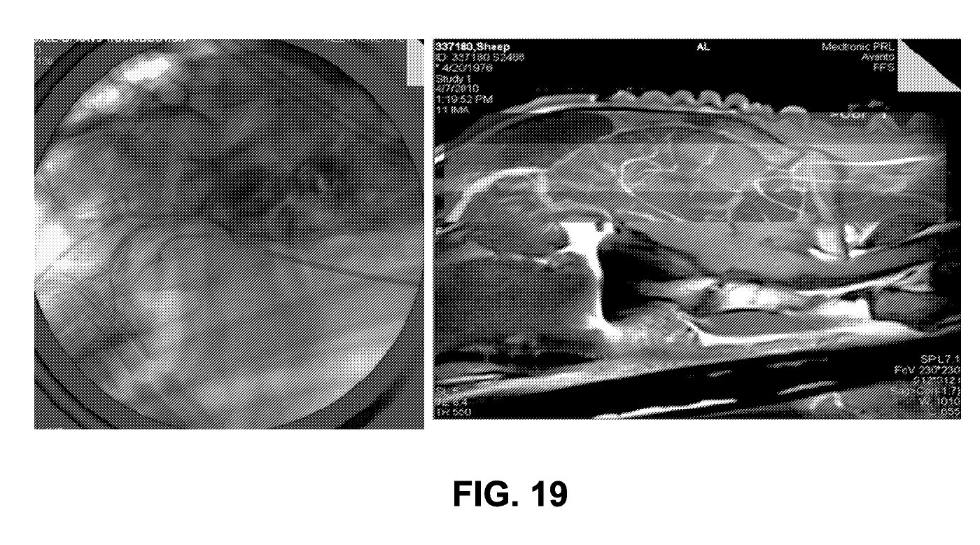

An effort was made to positively identify the point of the delivery of the viral vector in the arterial system of the sheep, by comparing the fluoroscopic images taken during the positioning of the guide catheter to T1 and time-of-flight (TOF) MRI images taken immediately post-operatively, which are reproduced in FIG. 19. While it was possible to superimpose the TOF and T1 MRI images to visualize the vasculature in the sheep brain, it was not possible to definitively superimpose the fluoroscopic images onto the TOF MRI views of the vasculature. Therefore, the identity of the artery utilized, and the exact point of the viral delivery, could not be determined.

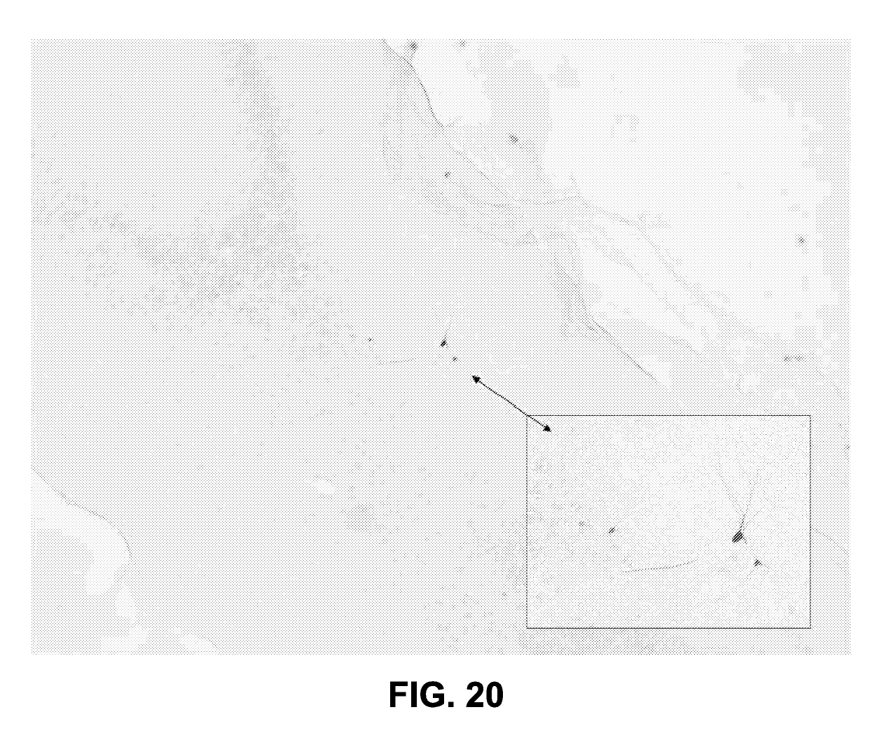

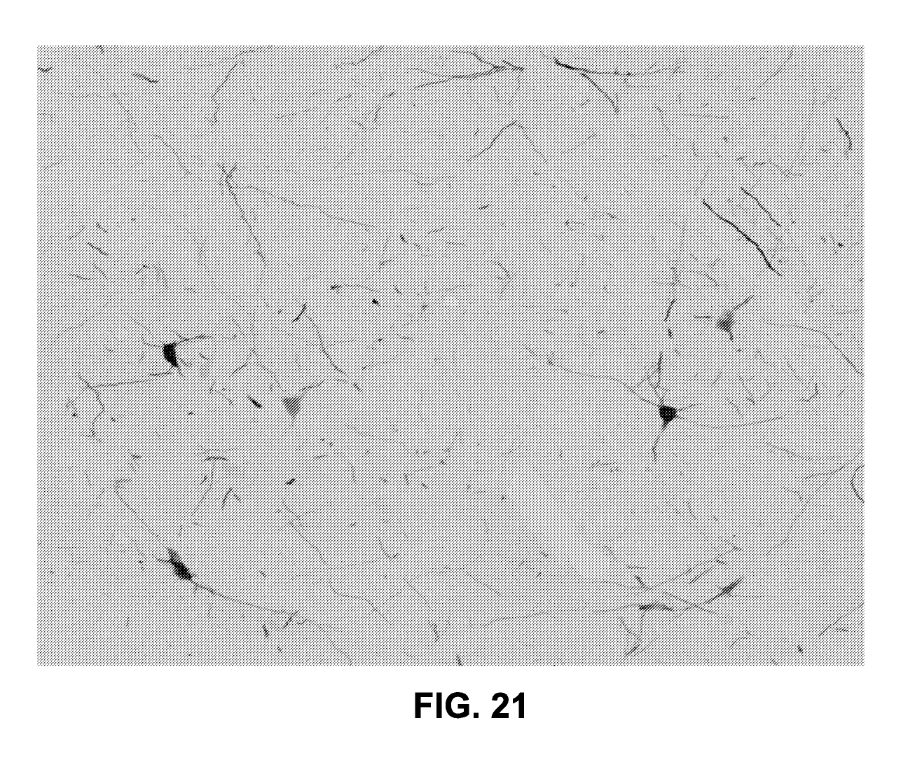

Twenty-three days post-viral delivery, the sheep was euthanized per standard protocol, and transcardially perfused with saline and 4% paraformaldehyde. The brain was removed and submitted to Neuroscience Associates for sectioning and staining for eGFP, visualized with DAB and nickle-DAB. Microscopic examination of the stained tissue sections revealed an occasional eGFP-positive cell, identified by cellular morphology, in the cerebral cortex of the sheep. In FIG. 20, e.g., occasional eGFP positive neurons are shown in the cerebral cortex of sheep receiving scAAV9 via cerebral artery infusion. However, the fact that these cells were few and far between makes it difficult to confidently conclude that these cells are evidence of passage of the scAAV9-eGFP into the brain across the blood-brain barrier. A greater density of stained cells was seen in the hindbrain of the sheep. In FIG. 21, e.g., eGFP positive cells are shown in the hindbrain of sheep receiving scAAV9 via cerebral artery infusion.

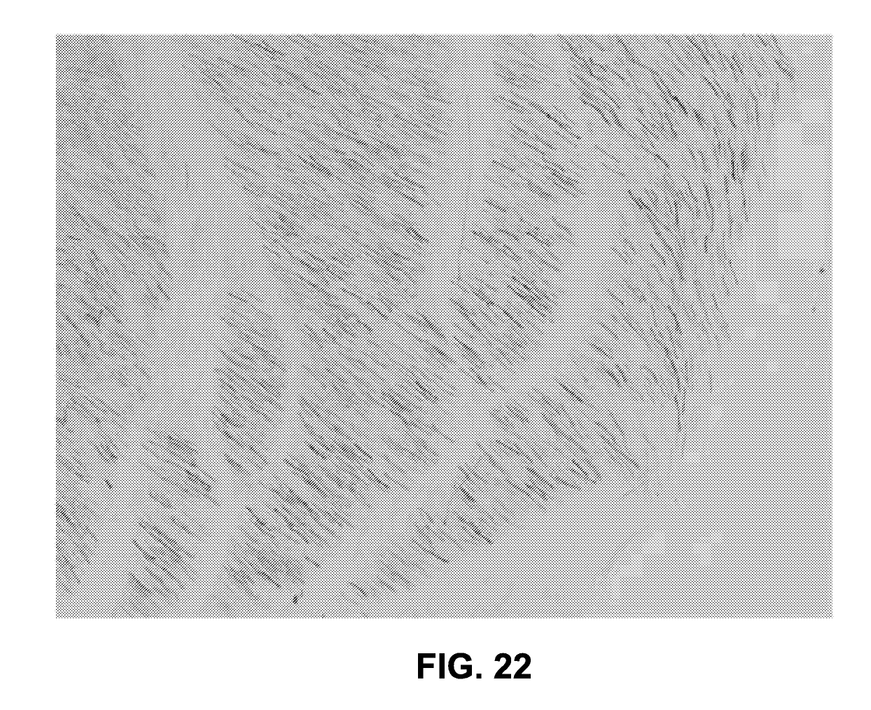

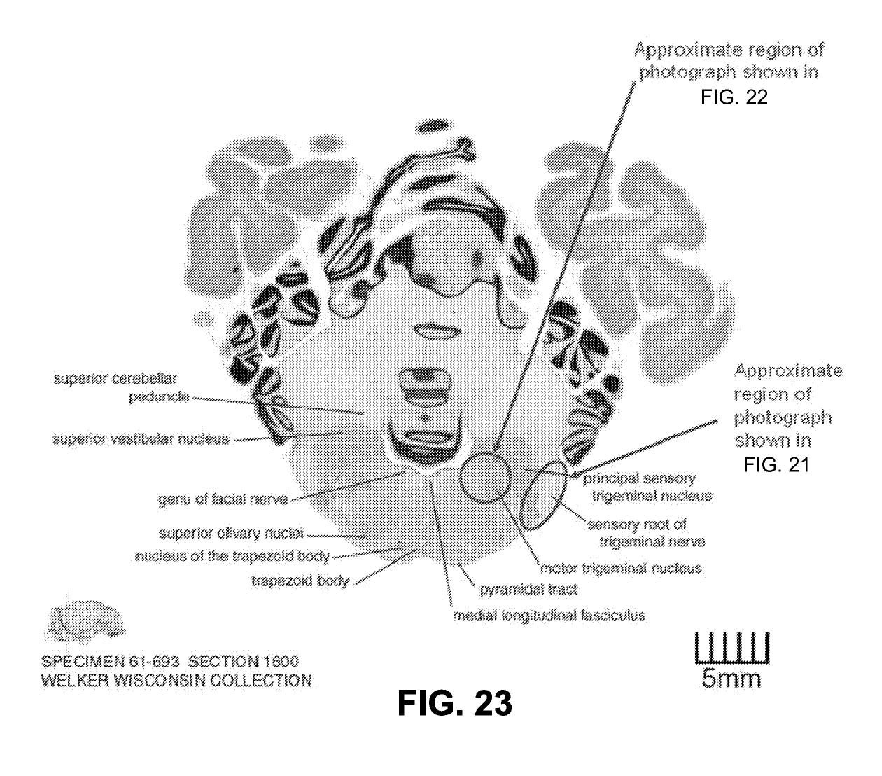

Adjacent to this cluster of stained cells shown in FIG. 21, a dense and anatomically delineated region of stained fibers was visible. FIG. 22 shows eGFP positive fibers in the hindbrain of sheep receiving scAAV9 via cerebral artery infusion. This observation suggested that neurons elsewhere were giving rise to this signal, with eGFP protein being distributed from the cell bodies into the axons, resulting in the eGFP staining seen in the fibers. However, no comparably numerous number of stained cells could be found elsewhere in the brain of the sheep. By comparison to a sheep brain atlas, e.g. the sheep brain atlas retrieved from https://msu.edu/˜brains/brains/sheep/scans/1600/image2.html and reproduced in FIG. 23, it may be hypothesized that the stained cluster of cells seen in the sheep hindbrain in FIG. 21 and the stained fiber tracts shown in FIG. 22 are the sheep's motor trigeminal nucleus and fibers of the trigeminal sensory tract, respectively.

Based on the foregoing observations, it is possible that the catheter tip position at the time of the delivery of the scAAV9-eGFP in this sheep was such that at least a portion of the scAAV9 was delivered to an artery serving the facial region of the sheep, including the trigeminal nerve, rather than to the brain. How much of the infusion of the scAAV9 went in this direction, rather than into an artery serving the cerebrum of the sheep (e.g., the middle cerebral artery) cannot be determined. Therefore, based on this one-animal experiment, the ability of scAAV9 to cross the blood-brain barrier in the sheep cannot be definitively established, nor definitively ruled-out. However, it can be concluded that a dose of 3.75×10e12 vg is insufficient to produce transduction of more than a few isolated cells in the brain upon circulation of the viral load throughout the circulatory system of the sheep.

For purposes of comparison to historical data concerning AAV distribution in sheep brain following an intraparenchymal injection, the scAAV9-eGFP was delivered to the second sheep via intraparenchymal injection not to the hippocampus of the sheep, but to the putamen.

Delivery to the putamen was done using the Medtronic Acute Neurological Therapy Infusion System, known as MANTIS. Targeting of the putamen was accomplished per the standard protocol for stereotactic burr hole surgery in the sheep, using the Stealth Station. A total of 135 μl of scAAV9-eGFP (lot V1084) at 1.12×10e12 vg/mL was delivered at 5 μl/minute, resulting in delivery of 1.5×10e11 vg. This volume, titer, and rate was chosen to be comparable to another sheep in a series of sheep receiving AAV1 in a previous study; the volume of distribution achieved in this previous sheep was among the best in the series.

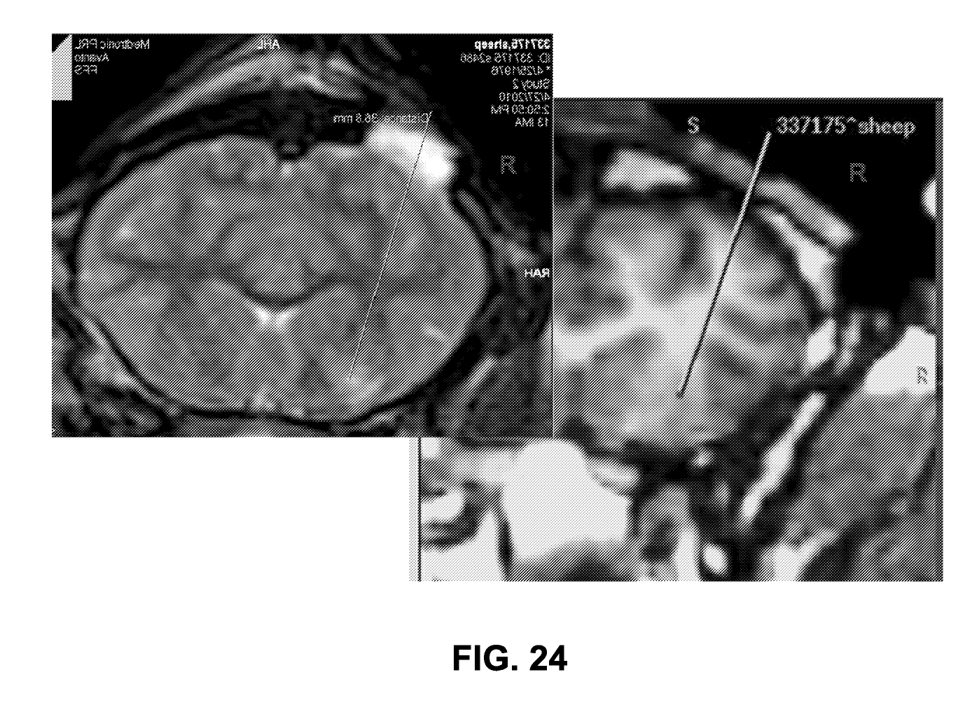

A post-operative MRI was taken to confirm successful catheter placement and fluid delivery. FIG. 24 compares the planned catheter path (Stealth Station, right panel) and post-operative MRI (left panel) in sheep receiving scAAV9 into the right putament via MANTIS. (The MRI image has been flipped on the vertical axis so that the right hemisphere is on the right side of the image, as in the Stealth Station snapshot.) The path length calculated by the Stealth Station from entry point to the putamen target was 36.8 mm; a line of this length graphically added to the MRI image is in good agreement with an entry point in the MANTIS anchor (appearing as the white artifact on the T2 MRI image) and a point of apparent fluid concentration in the T2 image in the sheep's right putamen.

As with the prior sheep, this sheep was terminated 23 days post-vector delivery and the brain was sectioned and stained for eGFP protein by Neuroscience Associates. The catheter path and point of vector delivery could not be definitively identified on the tissue sections, perhaps due to the small diameter of the MANTIS catheter. However, the staining results and the agreement between the MRI and Stealth Station images are consistent with accurate targeting and delivery of the vector into the right putamen.

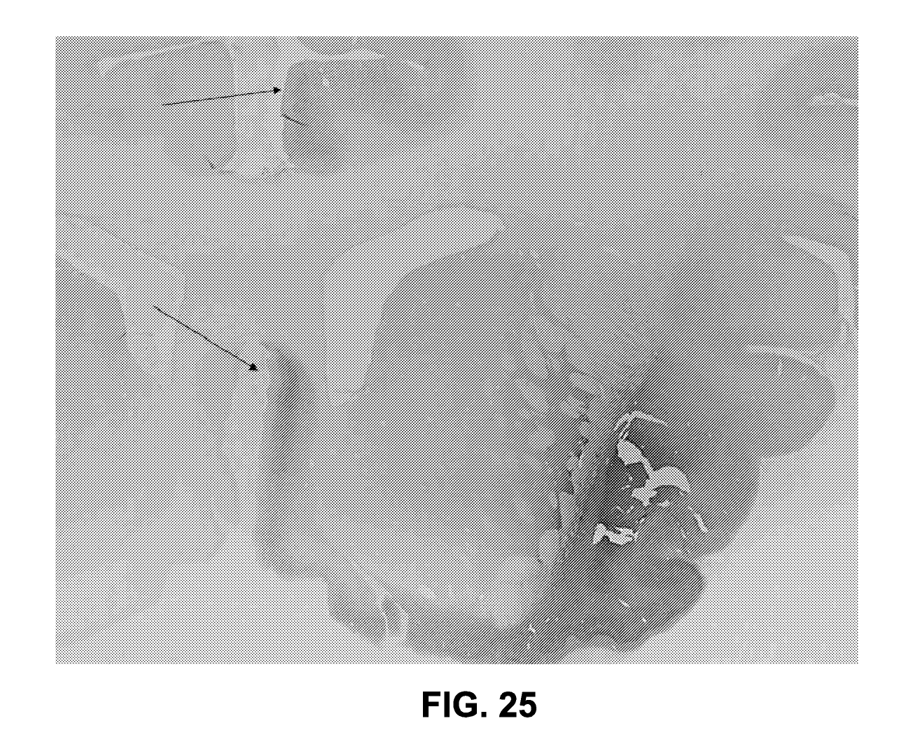

FIG. 25 is a coronal section of the brain of the second sheep receiving scAAV9-eGFP via direct infusion into the right putamen using MANTIS, immunostained for eGFP and visualized via DAB FIG. FIG. 25 shows the immunostaining for eGFP expression in the right putamen of the sheep.

The rostral-caudal extent of the eGFP staining seen in this animal spanned 18 sections; at 960 microns per section, the rostral-caudal extent was 17.28 mm. To further quantify the volume of distribution of the eGFP, the slides of the tissue were scanned into digital images (at 720 pixels per inch). The images were thresholded into binary images, using a threshold selected once manually, then applied to all images. Independently, the right putamen was manually outlined in each image (using NIH Image J software, public domain version 1.42q), and the contained area converted to a binary image. Finally, the software was used to AND the binary image of eGFP positive pixels and the binary image of the putamen, to yield a count of eGFP positive pixels in the putamen. These results were converted to mm2 area, and summed across 0.960 mm sections, to yield a total area in mm3. The results are summarized below in Table 3.

TABLE 3

| | | | | | | percent |
|---|---|---|---|---|---|---|
| | | | | | eGFP | “coverage” |
| | | | eGFP | | positive | of the |
| | Delivery | | positive | putamen | tissue within | putamen by |
| Sheep and | catheter and | Viral load | tissue | volume | the putamen | the |
| Hemisphere | rate | delivered | (mm3) | (mm3) | (mm3) | vector |
| (337175) | MANTIS | scAAV9 | 2282 | 393 | 345 | 88% |
| Right | 5 μl/min | 1.5 × 10¹¹ | | | | |
| Putamen | | | | | | |

By way of historical comparison, the results obtained in sheep in a previous study (S1297) using “standard” AAV serotype 1 are summarized below in Table 4.

TABLE 4

| | | | | | | percent |
|---|---|---|---|---|---|---|
| | | | | | eGFP | “coverage” |
| | | | eGFP | | positive | of the |
| | Delivery | | positive | putamen | tissue within | putamen by |
| Sheep and | catheter and | Viral load | tissue | volume | the putamen | the |
| Hemisphere | rate | delivered | (mm3) | (mm3) | (mm3) | vector |
| # 5 (330609) | Modified | AAV1 | 334 | 298 | 107 | 36% |
| Left | Medtronic | 1.5 × 10¹¹ | | | | |
| Putamen | Model 10640 | | | | | |
| | 5 μl/min | | | | | |
| # 5 (330609) | Modified | AAV1 | 257 | 330 | 95 | 29% |
| Right | Medtronic | 1.5 × 10¹¹ | | | | |
| Putamen | Model 8910 | | | | | |
| | 5 μl/min | | | | | |
| # 6 (330763) | Modified | AAV1 | 114 | 246 | 20.5 | 8% |
| Left | Medtronic | 1.5 × 10¹¹ | | | | |
| Putamen | Model 10640 | | | | | |
| | 5 μl/min | | | | | |
| # 7 (330532) | Modified | AAV1 | 198 | 547 | 57 | 10% |
| Right | Medtronic | 1.1 × 10¹¹ | | | | |
| Putamen | Model 10640 | | | | | |
| | 5 μl/min | | | | | |

When comparing the current results with the historical results from study S1297, several differences in methodology must be kept in mind. The current study utilized the MANTIS catheter (single end hole), rather than the modified Model 10640 used in study S1297 (a catheter with laser-drilled holes arranged radially around the catheter circumference at 120 degree intervals and along several centimeters of tip length). Also, the current study utilized NIH Image J software to quantify the pixel areas, rather than the MATLAB script used to process Photoshop stacked images in study S1297. Most importantly, the current study utilized a sensitive immunostain to visualize the eGFP protein, rather than fluorescence microscopy. Nevertheless, the substantial volume of distribution achieved using MANTIS-delivered scAAV9, including nearly complete coverage of the putamen, suggests that scAAV9 may be a particularly effective viral vector for delivery of DNA to this region of the brain. Note that the 88% coverage achieved is 4.7 standard deviations above the mean coverage (20.75%±13.89%) achieved in the prior study by comparable infusions of viral vector.

It is also notable that, as in the mouse studies using scAAV9, transduced cells were found in the sheep brain remote from the site of the delivery of the vector. FIG. 25 shows eGFP positive cells in the medial septal nucleus and cingulated cortex of the ipsilateral hemisphere of the sheep; these cells are located in areas such that the transduction of these cells probably cannot be accounted for by “leakage” of the viral vector up the catheter delivery track.

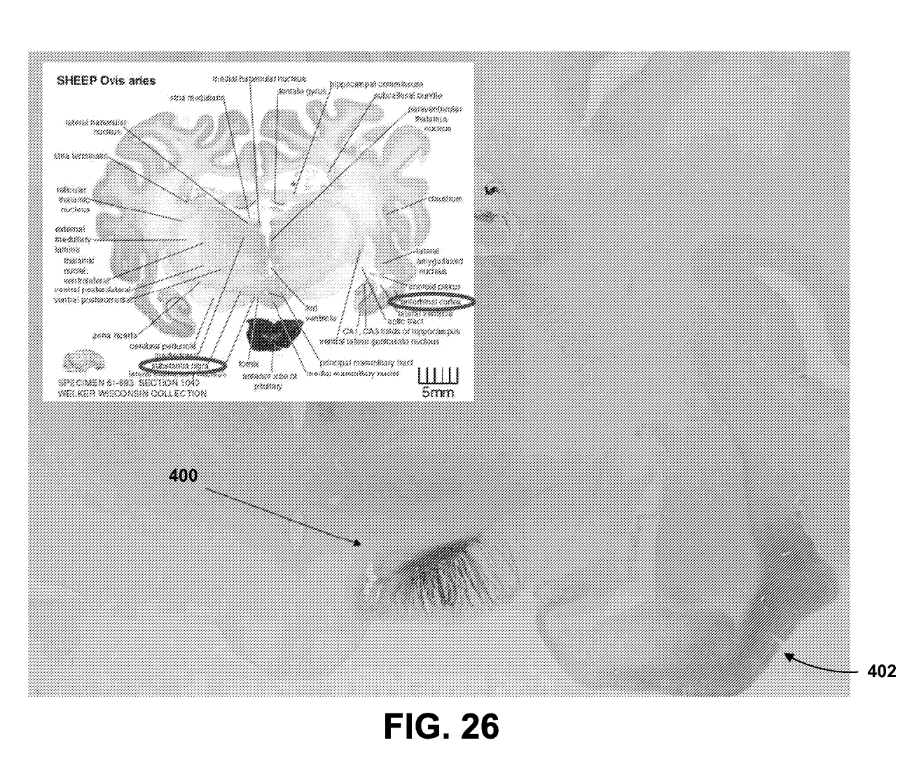

FIG. 26 provides a more dramatic example of transduction of cells remote from the infusion site. In this case, the cells and fibers are in the ipsilateral substantia nigra 400, indictative of likely retrograde transport of the viral vector from nigrostriatal terminals in the putamen back to the cell bodies. This tissue section also shows eGFP protein staining in the entrorhinal cortex 402 (also shown in atlas in the inset in FIG. 26).

Various examples have been described. These and other examples are within the scope of the following claims.

1. A method comprising:
delivering a viral vector comprising a genetic agent encoding for one or more light-sensitive proteins to a delivery site within a patient, wherein the viral vector comprises at least one of retrograde or anterograde transport properties such that the viral vector is configured to transduce the genetic agent into cells at the delivery site and into cells in a plurality of sites proximal and remote to the delivery site;
sensing a bioelectrical signal related to a neurological condition of the patient; and
delivering optical stimulation to one or more cells transduced with the genetic agent by the viral vector based on the sensed bioelectrical signal.

2. The method of claim 1, wherein delivering the viral vector comprises at least one of delivering the viral vector to the delivery site intravenously or delivering the viral vector to the delivery site intracranially.

3. The method of claim 1, wherein the viral vector comprises at least one of adeno-associated virus (AAV), herpes simplex virus (HSV), or lentivirus.

4. The method of claim 1, wherein the viral vector comprises at least one of a single-stranded nucleic acid or a self-complementary nucleic acid viral vector.

5. The method of claim 1, wherein the viral vector comprises at least one of AAV serotype 9 or AAV serotype 2.

6. The method of claim 1, wherein the one or more light-sensitive proteins comprise at least one of Channelrhodopsin-2 (ChR2), halorhodopsin (NpHR), archaerhodopsin-3 from Halorubrum sodomense (Arch), archaerhodopsin from Halorubrum strain TP009 (ArchT), or a blue-green light-drivable proton pump from the fungus Leptosphaeria maculans (Mac).

7. The method of claim 1, wherein the bioelectrical signal comprises at least one of a local field potential (LFP) of tissue of the patient, a signal associated with an electrocorticography (ECoG) of the brain of the patient, or a signal associated with an electroencephalography (EEG) of the brain of the patient.

8. The method of claim 1, wherein delivering the viral vector comprises delivering the viral vector to a delivery site within the brain of the patient.

9. The method of claim 8, wherein the viral vector comprises at least one of retrograde or anterograde transport properties such that the viral vector is configured to transduce the genetic agent into cells at the delivery site and into cells in a plurality of ipsilateral and contralateral sites within the brain of the patient.

10. The method of claim 1, wherein delivering the viral vector comprises delivering the viral vector to the hippocampus within a first hemisphere of the brain of the patient.

11. The method of claim 10, wherein the viral vector comprises at least one of retrograde or anterograde transport properties such that the viral vector is configured to transduce the genetic agent into cells in the hippocampus in the first hemisphere of the brain of the patient and into cells in a plurality of ipsilateral and contralateral sites within the brain of the patient.

12. The method of claim 11, wherein the plurality of ipsilateral and contralateral sites within the brain of the patient comprises at least one of the hippocampus in a second hemisphere of the brain of the patient or the cerebral cortex of the brain of the patient.

13. The method of claim 1, wherein delivering optical stimulation comprises delivering light to one or more of the cells transduced with the genetic agent by the viral vector, wherein the light is configured to activate at least one of the plurality of light-sensitive proteins in the one or more of the cells.

14. The method of claim 13, wherein delivering the light to the one or more of the cells transduced with the genetic agent by the viral vector comprises delivering at least one of a visible light comprising a wavelength in a range between from 380 nm to about 750 nm, an infrared light comprising a wavelength in a range between from 700 nm to about 300 μm, or an ultraviolet light comprising a wavelength from about 10 nm to about 400 nm.

15. The method of claim 13, wherein delivering the light to the one or more of the cells transduced with the genetic agent by the viral vector comprises delivering a light comprising a wavelength in a range from about 420 nm to about 500 nm.

16. The method of claim 15, wherein delivering the light to the one or more of the cells transduced with the genetic agent by the viral vector comprises delivering a light comprising a wavelength in a range from about 450 nm to about 495 nm.

17. The method of claim 16, wherein delivering the light to the one or more of the cells transduced with the genetic agent by the viral vector comprises delivering a light comprising a wavelength equal to about 470 nm.

18. The method of claim 13, wherein delivering the light to the one or more of the cells transduced with the genetic agent by the viral vector comprises delivering a light comprising a wavelength in a range from about 495 nm to about 570 nm.

19. The method of claim 18, wherein delivering the light to the one or more of the cells transduced with the genetic agent by the viral vector comprises delivering a light comprising a wavelength in a range from about 510 nm to about 550 nm.

20. The method of claim 19, wherein delivering the light to the one or more of the cells transduced with the genetic agent by the viral vector comprises delivering a light comprising a wavelength equal to about 535 nm.

21. The method of claim 13, wherein delivering the light to the one or more of the cells transduced with the genetic agent by the viral vector comprises delivering a light comprising a wavelength in a range from about 550 nm to about 610 nm.

22. The method of claim 21, wherein delivering the light to the one or more of the cells transduced with the genetic agent by the viral vector comprises delivering a light comprising a wavelength in a range from about 570 nm to about 590 nm.

23. The method of claim 22, wherein delivering the light to the one or more of the cells transduced with the genetic agent by the viral vector comprises delivering a light comprising a wavelength equal to about 580 nm.

24. The method of claim 1, wherein delivering optical stimulation comprises delivering a first light comprising a first wavelength to one or more of the cells transduced with the genetic agent by the viral vector and delivering a second light comprising a second wavelength to one or more of the cells transduced with the genetic agent by the viral vector.

25. The method of claim 24, wherein the first wavelength is different than the second wavelength.

26. The method of claim 24, wherein the first light is configured to activate at least one of the plurality of light-sensitive proteins in the one or more of the cells and the second light is configured to activate at least one other of the plurality of light-sensitive proteins in the one or more of the cells.

27. A medical system comprising:
a biological vector delivery device configured to deliver a viral vector comprising a genetic agent encoding for one or more light-sensitive proteins to a delivery site within a patient, wherein the viral vector comprises at least one of retrograde or anterograde transport properties such that the viral vector is configured to transduce the genetic agent into cells at the delivery site and into cells in a plurality of sites proximal and remote to the delivery site;
a sensor configured to sense a bioelectrical signal related to a neurological condition of the patient; and
an optical stimulator configured to deliver light to one or more of the cells transduced with the genetic agent by the viral vector based on the bioelectrical signal sensed by the sensor.

28. The system of claim 27, wherein the optical stimulator comprises:
at least one light source configured to generate light; and
at least one optical fiber connected to the light source and configured to deliver the light generated by the light source to the one or more of the cells transduced with the genetic agent by the viral vector; and
a processor configured to control the light source to generate light configured to activate at least one of the plurality of light-sensitive proteins in the one or more of the cells transduced with the genetic agent by the viral vector.

29. The system of claim 28, further comprising a plurality of optrodes connected to the at least one optical fiber and configured to deliver the at least one light generated by the light source to cells in one or more locations corresponding to the respective locations of the optrodes.

30. The system of claim 29, wherein the at least one optical fiber comprises a plurality of optical fibers connected to the plurality of optrodes such that each of the optrodes is configured to deliver the at least one light generated by the light source to one or more of the cells transduced with the genetic agent by the viral vector.

31. The system of claim 29, wherein the at least one optical fiber comprises a plurality of optical fibers connected to the plurality of optrodes such that each of a plurality of groups of the plurality of optrodes is configured to deliver the at least one light generated by the light source to one or more of the cells transduced with the genetic agent by the viral vector.

32. The system of claim 27, wherein the biological vector delivery device is configured to deliver the viral vector to at least one of delivering the viral vector to the delivery site intravenously or delivering the viral vector to the delivery site intracranially.

33. The system of claim 27, wherein the biological vector delivery device comprises a catheter configured to inject the viral vector into the brain of the patient intracranially.

34. The system of claim 27, wherein the viral vector comprises at least one of adeno-associated virus (AAV), herpes simplex virus (HSV), or lentivirus.

35. The system of claim 27, wherein the viral vector comprises at least one of a single-stranded nucleic acid or a self-complementary nucleic acid viral vector.

36. The system of claim 27, wherein the viral vector comprises at least one of AAV serotype 9 or AAV serotype 2.

37. The system of claim 27, wherein the one or more light-sensitive proteins comprise at least one of Channelrhodopsin-2 (ChR2), halorhodopsin (NpHR), archaerhodopsin-3 from Halorubrum sodomense (Arch), archaerhodopsin from Halorubrum strain TP009 (ArchT), or a blue-green light-drivable proton pump from the fungus Leptosphaeria maculans (Mac).

38. The system of claim 27, wherein the sensor comprises an electrode configured to sense at least one of a local field potential (LFP) of tissue of the patient, a signal associated with an electrocorticography (ECoG) of the brain of the patient, or a signal associated with an electroencephalography (EEG) of the brain of the patient.

39. The system of claim 27, wherein the biological vector delivery device is configured to deliver the viral vector to a delivery site within the brain of the patient.

40. The system of claim 39, wherein the viral vector comprises at least one of retrograde or anterograde transport properties such that the viral vector is configured to transduce the genetic agent into cells at the delivery site and into cells in a plurality of ipsilateral and contralateral sites within the brain of the patient.

41. The system of claim 27, wherein the biological vector delivery device is configured to deliver the viral vector to the hippocampus within a first hemisphere of the brain of the patient.

42. The system of claim 41, wherein the viral vector comprises at least one of retrograde or anterograde transport properties such that the viral vector is configured to transduce the genetic agent into cells in the hippocampus in the first hemisphere of the brain of the patient and into cells in a plurality of ipsilateral and contralateral sites within the brain of the patient.

43. The system of claim 42, wherein the plurality of ipsilateral and contralateral sites within the brain of the patient comprises at least one of the hippocampus in a second hemisphere of the brain of the patient or the cerebral cortex of the brain of the patient.

44. A system comprising:
means for delivering a viral vector comprising a genetic agent encoding for one or more light-sensitive proteins to a delivery site within a patient, wherein the viral vector comprises at least one of retrograde or anterograde transport properties such that the viral vector is configured to transduce the genetic agent into cells at the delivery site and into cells in a plurality of sites proximal and remote to the delivery site;
means for sensing a bioelectrical signal related to a neurological condition of the patient; and
means for delivering optical stimulation to one or more cells transduced with the genetic agent by the viral vector based on the bioelectrical signal.

45. A method comprising:
delivering a viral vector comprising a genetic agent encoding for one or more light-sensitive proteins to a delivery site in the hippo campus within a first hemisphere of the brain of a patient, wherein the viral vector comprises at least one of retrograde or anterograde transport properties such that the viral vector is configured to transduce the genetic agent into cells at the delivery site and into cells in a plurality of sites proximal and remote to the delivery site;
sensing a bioelectrical signal related to epilepsy; and
delivering optical stimulation to one or more cells transduced with the genetic agent in the cerebral cortex of the brain of the patient based on the sensed bioelectrical signal.

46. A medical system comprising:
a biological vector delivery device configured to deliver a viral vector comprising a genetic agent encoding for one or more light-sensitive proteins to a delivery site in the hippocampus within a first hemisphere of the brain of a patient, wherein the viral vector comprises at least one of retrograde or anterograde transport properties such that the viral vector is configured to transduce the genetic agent into cells at the delivery site and into cells in a plurality of sites proximal and remote to the delivery site;
a sensor configured to sense a bioelectrical signal related to epilepsy; and
an optical stimulator configured to deliver light to one or more of the cells transduced with the genetic agent in the cerebral cortex of the brain of the patient based on the bioelectrical signal sensed by the sensor.


SEQUENCE LISTING (3 sequences)

SEQ ID NO: 1 - DNA, 930 bp, Chlamydomonas reinhardtii
atggattatggaggcgccctgagtgccgttgggcgcgagctgctatttgtaacgaacccagtagtcgtcaatggctctgtacttgtgcctgaggaccagtgttactgcgcgggctggattgagtcgcgtggcacaaacggtgcccaaacggcgtcgaacgtgctgcaatggcttgctgctggcttctccatcctactgcttatgttttacgcctaccaaacatggaagtcaacctgcggctgggaggagatctatgtgtgcgctatcgagatggtcaaggtgattctcgagttcttcttcgagtttaagaacccgtccatgctgtatctagccacaggccaccgcgtccagtggttgcgttacgccgagtggcttctcacctgcccggtcattctcattcacctgtcaaacctgacgggcttgtccaacgactacagcaggcgcaccatgggtctgcttgtgtctgatattggcacaattgtgtggggcgccacttccgccatggccaccggatacgtcaaggtcatcttcttctgcctgggtctgtgttatggtgctaacacgttctttcacgctgccaaggcctacatcgagggttaccacaccgtgccgaagggccggtgtcgccaggtggtgactggcatggcttggctcttcttcgtatcatggggtatgttccccatcctgttcatcctcggccccgagggcttcggcgtcctgagcgtgtacggctccaccgtcggccacaccatcattgacctgatgtcgaagaactgctggggtctgctcggccactacctgcgcgtgctgatccacgagcatatcctcatccacggcgacattcgcaagaccaccaaattgaacattggtggcactgagattgaggtcgagacgctggtggaggacgaggccgaggctggcgcggtaccc

SEQ ID NO: 2 - DNA, 930 bp, Chlamydomonas reinhardtii
atggactatggcggcgctttgtctgccgtcggacgcgaacttttgttcgttactaatcctgtggtggtgaacgggtccgtcctggtccctgaggatcaatgttactgtgccggatggattgaatctcgcggcacgaacggcgctcagaccgcgtcaaatgtcctgcagtggcttgcagcaggattcagcattttgctgctgatgttctatgcctaccaaacctggaaatctacatgcggctgggaggagatctatgtgtgcgccattgaaatggttaaggtgattctcgagttcttttttgagtttaagaatccctctatgctctaccttgccacaggacaccgggtgcagtggctgcgctatgcagagtggctgctcacttgtcctgtcatccttatccacctgagcaacctcaccggcctgagcaacgactacagcaggagaaccatgggactccttgtctcagacatcgggactatcgtgtggggggctaccagcgccatggcaaccggctatgttaaagtcatcttcttttgtcttggattgtgctatggcgcgaacacattttttcacgccgccaaagcatatatcgagggttatcatactgtgccaaagggtcggtgccgccaggtcgtgaccggcatggcatggctgtttttcgtgagctggggtatgttcccaattctcttcattttggggcccgaaggttttggcgtcctgagcgtctatggctccaccgtaggtcacacgattattgatctgatgagtaaaaattgttgggggttgttgggacactacctgcgcgtcctgatccacgagcacatattgattcacggagatatccgcaaaaccaccaaactgaacatcggcggaacggagatcgaggtcgagactctcgtcgaagacgaagccgaggccggagccgtgcca

SEQ ID NO: 3 - DNA, 924 bp, Natronomonas pharaonis
atgaggggtacgcccctgctcctcgtcgtctctctgttctctctgcttcaggacacagagaccctgcctcccgtgaccgagagtgccgtggcccttcaagccgaggttacccaaagggagttgttcgagttcgtgctgaacgaccctttgcttgcaagcagtctctatatcaacatcgcacttgcaggactgagtatactgctgttcgtttttatgacccgaggactcgatgatccacgggcaaaacttattgctgtgtcaaccatccttgtgcctgtcgtcagcattgcctcctacactggattggcgagcggcctgacaatttccgttcttgaaatgccagcgggccattttgcagaaggcagctcagtgatgctgggaggagaagaggtagatggtgtagtcaccatgtggggacggtatctcacctgggcactttccacgcccatgattctcctcgctctgggtctcctggccggaagcaatgctacaaagctcttcacagctatcactttcgatatcgctatgtgcgtgactggccttgccgcggccctgactacctcctcccacctcatgagatggttctggtacgctatcagttgtgcatgctttctggtggtcttgtatatcctgctggtggagtgggcacaggacgccaaagccgcgggaaccgctgacatgttcaataccctgaagctgttgacagtagtgatgtggctggggtatccaattgtgtgggctcttggagtcgagggtatcgcggtgttgcccgttggggtgacgagctggggatattctttcctggatatcgtggcaaagtacattttcgcattcttgctcctgaactatctgacgtcaaacgaatctgtcgtgtccggcagcattttggatgttccatctgcttctgggaccccggctgatgat